(19) United States
(12) Patent Application Publication
(10) Pub. No.: US 2021/0137853 A1
(43) Pub. Date: May 13, 2021

(54) METHODS OF TREATING EPILEPTIC PATIENTS WITH FENFLURAMINE

(71) Applicant: Zogenix International Limited, Maidenhead (GB)

(72) Inventors: Gail M. Farfel, Franklin Lakes, NJ (US); Thadd Reeder, San Carlos, CA (US); Parthena M. Martin, Reno, NV (US); Glenn Morrison, Half Moon Bay, CA (US)

(21) Appl. No.: 17/024,573

(22) Filed: Sep. 17, 2020

Related U.S. Application Data
(60) Provisional application No. 63/044,932, filed on Jun. 26, 2020.
(60) Provisional application No. 62/901,514, filed on Sep. 17, 2019.

(51) Int. Cl. A61K 31/137; A61K 31/451; A61K 31/5513; A61K 31/335; A61K 31/554; A61K 31/4535; A61K 31/5415; A61K 31/55; A61K 31/519; A61K 31/36; A61K 31/19; A61P 25/08; A61K 31/454; A61K 31/553; A61K 31/404; A61K 31/438; A61K 31/495; A61K 31/53

(52) U.S. Cl. CPC A61K 31/137; A61K 31/53; A61K 31/5513; A61K 31/335; A61K 31/554; A61K 31/4535; A61K 31/5415; A61K 31/55; A61K 31/519; A61K 31/36; A61K 31/19; A61P 25/08; A61K 31/454; A61K 31/553; A61K 31/404; A61K 31/438; A61K 31/495; A61K 31/451

(57) ABSTRACT

The present disclosure provides methods of treating and/or preventing symptoms of epilepsy or epileptic encephalopathy where fenfluramine or a pharmaceutically acceptable salt thereof is or has been administered to a patient or population of patients. The present disclosure encompasses a recognition of contraindication of fenfluramine and certain serotonin receptor agonists, particularly a CNS penetrant serotonin receptor antagonist. The present disclosure provides methods where said patients being administered fenfluramine have been warned against co-administration of certain serotonin receptor antagonists, are not co-administered a serotonin receptor antagonist, and/or in which co-administration of a serotonin-receptor antagonist is discontinued.

### I. FIELD

This invention relates generally to the field of treating epilepsy and/or epileptic encephalopathy with fenfluramine, and the discovery that co-administration of certain serotonin receptor antagonists can cause a loss of antiseizure effect.

### II. BACKGROUND

Epilepsy is a central nervous system (neurological) disorder characterized by seizures or periods of unusual behavior, sensations, and sometimes loss of awareness. Fenfluramine, i.e. 3-trifluoromethyl-N-ethylamphetamine, is an amphetamine derivative having a number of therapeutic uses including treatment of certain forms of epilepsy and/or epileptic encephalopathy. There exists a continuing need for safe and effective therapies with fenfluramine that take into account contraindications with other drugs.

### III. SUMMARY

The present disclosure provides the insight that certain serotonergic antagonists can impair the anti-seizure effect of fenfluramine. The present disclosure encompasses a recognition that some patients being administered and/or exposed to fenfluramine may develop extreme weight loss, wasting, cachexia and/or loss of appetite, such that co-administration of an appetite stimulant is recommended in these patients. Among other things, the present disclosure recognizes a problem with certain appetite stimulants that are serotonergic antagonists, in that certain serotonergic antagonists may reduce efficacy of fenfluramine.

In some embodiments, the present disclosure provides methods and kits whereby fenfluramine-treated patients (e.g., those that develop extreme weight loss, wasting, cachexia and/or loss of appetite) (i) are warned against co-administration of certain serotonin receptor antagonists (e.g., are informed that co-administration of certain serotonin receptor antagonists may decrease fenfluramine's protection against seizures), (ii) are not co-administered a serotonin receptor antagonist, and/or (iii) in which co-administration of a serotonin-receptor antagonist is discontinued.

In some embodiments, the present disclosure provides methods of treating epilepsy comprising: administering a therapeutically effective amount of fenfluramine to a patient in need thereof, where said patient is not concurrently being administered and/or exposed to a serotonin (5-HT)-receptor antagonist.

In some embodiments, a patient is also characterized by extreme weight loss, wasting, cachexia and/or loss of appetite.

In some embodiments, a patient is also characterized by a co-morbid psychiatric condition and/or psychosis. In some embodiments, a co-morbid psychiatric condition or psychosis is or comprises tangential, incoherent speech and thought, hallucinations, delusions, and aggression, as well as exaggerated, bizarre, disorganized behaviors, poverty of speech or speech content, flattened affect, social withdrawal, anhedonia, apathy, impaired attention, and/or impaired self-monitoring.

In some embodiments, the present disclosure provides methods of treating epilepsy in a population of patients, wherein the patients have been diagnosed with epilepsy and/or epileptic encephalopathy, and wherein the patients are also characterized by extreme weight loss, wasting, cachexia and/or loss of appetite, the method comprising: administering a therapeutically effective amount of fenfluramine to those patients to those patients who are not being administered and/or exposed to a serotonin (5-HT)-receptor antagonist.

In some embodiments, the present disclosure provides methods of treating a population of patients diagnosed with epilepsy and/or epileptic encephalopathy, the method comprising: (i) administering a therapeutically effective amount of fenfluramine or a pharmaceutically acceptable salt thereof, (ii) monitoring the patients for extreme weight loss, wasting, cachexia and/or loss of appetite, and in those patients that develop extreme weight loss, wasting, cachexia and/or loss of appetite, (iii) administering an appetite stimulant, wherein the appetite stimulant is not a serotonin (5-HT)-receptor antagonist.

In some embodiments, fenfluramine is administered at a daily dose within a range of about 0.1 mg/kg/day to about 2.5 mg/kg/day. In some embodiments, fenfluramine is administered at a daily dose of less than about 2.5 mg/kg/day, less than about 2.0 mg/kg/day, less than about 1.5 mg/kg/day, or less than about 1.0 mg/kg/day, such as about 1.0 mg/kg/day, about 0.95 mg/kg/day, about 0.9 meg/kg/day, about 0.85 mg/kg/day, about 0.85 mg/kg/day, about 0.8 mg/kg/day, about 0.75 mg/kg/day, about 0.7 mg/kg/day, about 0.65 mg/kg/day, about 0.6 mg/kg/day, about 0.55 mg/kg/day, about 0.5 mg/kg/day, about 0.45 mg/kg/day, about 0.4 mg/kg/day, about 0.35 mg/kg/day, about 0.3 mg/kg/day, about 0.25 mg/kg/day, about 0.2 mg/kg/day, about 0.15 mg/kg/day to about 0.1 mg/kg/day.

In some embodiments, a therapeutically effective total daily dose of fenfluramine is no more than 40 mg, no more than 30 mg, or no more than 20 mg.

The present disclosure encompasses a recognition that in embodiments where fenfluramine is administered at a low dose (e.g., within a range of 0.1 mg/kg to 0.5 mg/kg, e.g., a dose of 0.1 mg/kg to 0.35 mg/kg twice daily), the antiseizure activity of fenfluramine may be susceptible to interference by other agents such as certain serotonin receptor antagonists. In certain embodiments, a dose of fenfluramine is increased when one or more certain serotonin-receptor antagonists are being co-administered.

In some embodiments, fenfluramine is co-administered with an anti-psychotic selected from: phenothiazines (trifluoperazine, perphenazine, prochlorperazine, acetophenazine, triflupromazine, mesoridazine), butyrophenones (haloperidol), thioxanthenes (chlorprothixene), dihydroindoles (molindone), diphenylbutylpiperidines (pimozide), risperidone, quetiapine, aripiprazole, paliperidone, cariprazine, brexpiprazole, and tricyclic antihistamines (cyproheptadine; pizotifen; ketotifen, azatadine, loratadine and desloratadine).

In some embodiments, the present disclosure provides methods of treating weight loss, wasting, cachexia and/or loss of appetite in a population of epileptic patients being administered and/or exposed to fenfluramine or a pharmaceutically acceptable salt thereof, the method comprising: administering an appetite stimulant that is not a serotonin (5-HT)-receptor antagonist.

In some embodiments, the present disclosure provides methods of treating weight loss, wasting, cachexia and/or loss of appetite in a population of epileptic patients being administered and/or exposed to fenfluramine or a pharmaceutically acceptable salt thereof, the method comprising: (i) administering a serotonin (5-HT)-receptor antagonist to the patients, and (ii) providing to the patient instructions that the anti-seizure efficacy of fenfluramine or a pharmaceutically acceptable salt thereof may be reduced by administration of said serotonin (5-HT)-receptor antagonist. In some embodiments, patients being administered and/or exposed to a serotonin (5-HT)-receptor antagonist are monitored for seizures (e.g., breakthrough seizures).

In some embodiments, patients that experience weight loss, wasting, and/or loss of appetite may be co-treated with an appetite stimulant that is, e.g., dronabinol (THC), megesterol and/or oxandrolone, Orexin (Vitamin B complex supplement), Benadryl [diphenhydramine], clemastine and chlopheniramine), fexofenadine and/or cetirizine.

In some embodiments, the present disclosure provides methods of treating psychosis in a population of epileptic patients being administered and/or exposed to fenfluramine or a pharmaceutically acceptable salt thereof, the method comprising: (i) administering a serotonin (5-HT)-receptor antagonist to the patients, and (ii) providing to the patient instructions that the anti-seizure efficacy of fenfluramine or a pharmaceutically acceptable salt thereof may be reduced by administration of said serotonin (5-HT)-receptor antagonist. In some embodiments, patients being administered and/or exposed to a serotonin (5-HT)-receptor antagonist are monitored for seizures (e.g., breakthrough seizures). In some embodiments, a psychosis is or comprises tangential, incoherent speech and thought, hallucinations, delusions, and aggression, as well as exaggerated, bizarre, disorganized behaviors, poverty of speech or speech content, flattened affect, social withdrawal, anhedonia, apathy, impaired attention, and/or impaired self-monitoring.

In some embodiments, the present disclosure provides methods of administering fenfluramine to an epilepsy patient, wherein said patient is also in need of treatment for another centrally mediated condition, said method comprising: (i) providing to the patient a therapeutically effective amount of fenfluramine or a pharmaceutically acceptable salt thereof, and (ii) providing to the patient instructions that the anti-seizure efficacy of fenfluramine or a pharmaceutically acceptable salt thereof may be reduced by administration of a serotonin receptor antagonist.

In some embodiments, the present disclosure provides methods of treating a symptom of epilepsy or epileptic encephalopathy in a patient diagnosed with epilepsy or epileptic encephalopathy, comprising: administering a therapeutically effective dose of fenfluramine or a pharmaceutically acceptable salt thereof to the patient, wherein the patient is not being treated with a 5-HT receptor antagonist.

In some embodiments, a therapeutically effective dose of fenfluramine is within a range of about 0.1 mg/kg/day to about 2.5 mg/kg/day. In some embodiments, a therapeutically effective dose of fenfluramine is a daily dose of less than about 2.5 mg/kg/day, less than about 2.0 mg/kg/day, less than about 1.5 mg/kg/day, or less than about 1.0 mg/kg/day, such as about 1.0 mg/kg/day, about 0.95 mg/kg/day, about 0.9 meg/kg/day, about 0.85 mg/kg/day, about 0.85 mg/kg/day, about 0.8 mg/kg/day, about 0.75 mg/kg/day, about 0.7 mg/kg/day, about 0.65 mg/kg/day, about 0.6 mg/kg/day, about 0.55 mg/kg/day, about 0.5 mg/kg/day, about 0.45 mg/kg/day, about 0.4 mg/kg/day, about 0.35 mg/kg/day, about 0.3 mg/kg/day, about 0.25 mg/kg/day, about 0.2 mg/kg/day, about 0.15 mg/kg/day to about 0.1 mg/kg/day.

In some embodiments, a therapeutically effective total daily dose of fenfluramine is no more than 40 mg, no more than 30 mg, or no more than 20 mg.

In some embodiments, a 5-HT receptor antagonist is selected from Table 3.

In some embodiments, a serotonin receptor antagonist is selected from: cyproheptadine, or a 5-HT_{1A} serotonin receptor antagonist, a 5-HT_{1D} serotonin receptor antagonist, a 5-HT_{2A} serotonin receptor antagonist, or a 5-HT_{2C} serotonin receptor antagonist.

In some embodiments, a serotonin receptor antagonist is a 5-HT_{1A} serotonin receptor antagonist and/or 5-HT_{2C} serotonin receptor antagonist. In some certain embodiments, a serotonin receptor antagonist is a 5-HT_{1A} serotonin receptor antagonist selected from: BRL 15572, MDL 73005 EF, N-desmethylclozapine, and ORG-5222. In some embodiments, a serotonin receptor antagonist is a 5-HT_{2C} serotonin receptor antagonist selected from: chlorpromazine, mianserin, perphenazine, and loxapin.

In certain embodiments, a patient is not being treated with a 5-HT_{1D} serotonin receptor antagonist or a 5-HT_{2A} serotonin receptor antagonist. In certain embodiments, a patient is informed that a 5-HT_{1D} serotonin receptor antagonist or a 5-HT_{2A} serotonin receptor antagonist may impair efficacy of fenfluramine (e.g., increased probability of seizures). In certain embodiments, a patient is not being treated with a 5-HT_{1D} serotonin receptor antagonist or a 5-HT_{2A} serotonin receptor antagonist selected from: BRL 15572, chlorpromazine, mianserin, N-desmethylclozapine, ORG-5222, perphenazine, loxapine, and pimozide.

In certain embodiments, a patient is treated with a 5-HT receptor antagonist selected from 5-HT_{1A} serotonin receptor antagonist and a 5-HT_{2C} serotonin receptor antagonist. In some embodiments, a patient that is informed that a 5-HT_{1D} serotonin receptor antagonist or a 5-HT_{2A} serotonin receptor antagonist may impair efficacy of fenfluramine is treated with a 5-HT_{1A} serotonin receptor antagonist and/or a 5-HT_{2C} serotonin receptor antagonist.

In some embodiments, the present disclosure provides methods of treating weight loss, wasting, cachexia and/or loss of appetite in a population of epileptic patients being administered and/or exposed to fenfluramine or a pharmaceutically acceptable salt thereof, the method comprising: (i) administering a 5-HT_{1A} serotonin receptor antagonist or a 5-HT_{2C} serotonin receptor antagonist to the patients, and (ii) providing to the patient instructions that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

In certain embodiments, a dose of fenfluramine is increased when one or more certain serotonin-receptor antagonists are being co-administered.

In some embodiments, the present disclosure provides methods of treating a population of patients diagnosed with epilepsy and/or epileptic encephalopathy, the method comprising: (i) administering a therapeutically effective amount of fenfluramine or a pharmaceutically acceptable salt thereof, (ii) monitoring the patients for extreme weight loss, wasting, cachexia and/or loss of appetite, and in those patients that develop extreme weight loss, wasting, cachexia and/or loss of appetite, (iii) administering an appetite stimulant, wherein the appetite stimulant is a 5-HT_{1A} serotonin receptor antagonist or a 5-HT_{2C} serotonin receptor antagonist.

In some embodiments, provided methods further include providing instructions that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

In some embodiments, epilepsy and/or epileptic encephalopathy in the context of the present disclosure is or comprises Dravet syndrome, Lennox Gastaut syndrome, Rett syndrome, Doose syndrome, West syndrome, Tuberous Sclerosis Complex (TSC), Dup15q Syndrome, CDKL5 Deficiency Disorder, epileptic encephalopathy associated with mutation(s) in the PCDH19 Gene, and/or an epileptic encephalopathies associated with mutations in sodium channel genes.

In some embodiments, epilepsy and/or epileptic encephalopathy in the context of the present disclosure is or comprises Dravet syndrome, Lennox Gastaut syndrome, Rett syndrome, Doose syndrome, and/or West syndrome.

In some embodiments, a serotonin receptor antagonist is a 5-HT_{1A} serotonin receptor antagonist and/or 5-HT_{2C} serotonin receptor antagonist that is selected from cyproheptadine, clozapine, doxepin, quetiapine, ketotifen, pizotifen, perphenazine, mianserin, mirtazapine, risperidone and asenapine.

In some embodiments, fenfluramine is administered or has been administered in a dose that is in a range of from 10.0 mg/kg/day to 0.01 mg/kg/day.

In some embodiments, fenfluramine is administered or has been administered in a dosage form selected from the group consisting of oral, injectable, transdermal, inhaled, nasal, buccal, rectal, vaginal and parenteral delivery.

In some embodiments, fenfluramine is administered or has been administered as an oral solution. In some certain embodiments, fenfluramine is administered or has been administered in an amount within a range of 10 mg to 200 mg. In some embodiments, fenfluramine is administered or has been administered in an amount that is 120 mg or less, 60 mg or less, 30 mg or less, and 20 mg or less.

In some embodiments, a therapeutically effective total daily dose of fenfluramine is no more than 40 mg, no more than 30 mg, or no more than 20 mg.

In some embodiments, provided methods further include administering a co-therapeutic agent selected from the group consisting of stiripentol, clobazam, and valproate.

In some embodiments, the present disclosure provides methods for treating epilepsy and/or epileptic encephalopathy, comprising: (i) providing a pharmaceutical composition comprising a therapeutically effective amount of fenfluramine or a pharmaceutically acceptable salt thereof and a pharmaceutically acceptable carrier; and (ii) advising the physician by a label, a product insert or medication guide accompanying the pharmaceutical composition that some patients may experience weight loss, wasting, and/or loss of appetite, and to avoid co-treatment with an appetite stimulant that is a serotonin receptor antagonist.

In some embodiments, the present disclosure provides methods for treating epilepsy and/or epileptic encephalopathy that is also characterized by psychosis, comprising: (i) providing a pharmaceutical composition comprising a therapeutically effective amount of fenfluramine or a pharmaceutically acceptable salt thereof and a pharmaceutically acceptable carrier; and (ii) advising the physician by a label, a product insert or medication guide accompanying the pharmaceutical composition to avoid co-treatment with an anti-psychotic that is a serotonin receptor antagonist.

In some embodiments, a serotonin receptor antagonist is cyproheptadine. In some embodiments, a serotonin receptor antagonist is a 5-HT_{1D} serotonin receptor antagonist or a 5-HT_{2A} serotonin receptor antagonist.

In some embodiments, patients that experience weight loss, wasting, and/or loss of appetite may be co-treated with an appetite stimulant that is, e.g., dronabinol (THC), megesterol and/or oxandrolone, Orexin (Vitamin B complex supplement), Benadryl [diphenhydramine], clemastine and chlopheniramine), fexofenadine and/or cetirizine.

In some embodiments, epilepsy and/or epileptic encephalopathy in the context of the present disclosure is or comprises Dravet syndrome, Lennox Gastaut syndrome, Rett syndrome, Doose syndrome, and/or West syndrome.

In some embodiments, the present disclosure provides methods comprising: treating a patient diagnosed with (a) Dravet syndrome or Lennox-Gastaut syndrome, and (b) weight loss, wasting, and/or loss of appetite, by providing a formulation comprising a pharmaceutically acceptable carrier and a therapeutically effective amount of fenfluramine or a pharmaceutically acceptable salt thereof; and advising the physician by a label, a product insert or medication guide accompanying the formulation to avoid co-treatment of the patient with a serotonin receptor antagonist.

In some embodiments, a serotonin receptor antagonist is cyproheptadine. In some embodiments, a serotonin receptor antagonist is a 5-HT_{1D} serotonin receptor antagonist or a 5-HT_{2A} serotonin receptor antagonist.

In some embodiments, a serotonin receptor antagonist is fluphenazine, thioridazine, thiothixene, flupenthixol, amoxapine, loxapine, olanzapine, ziprasidone, asenapine, lurasidone, iloperidone, clozapine, mianserin and mirtazapine.

In some embodiments, a serotonin receptor antagonist is a 5-HT_{1D} serotonin receptor antagonist selected from: ergotamine, lisuride, lysergol, metergoline, methiothepin, naratriptan, oxymetazoline, sumatriptan, and ziprasidone.

In some embodiments, a serotonin receptor antagonist is a 5-HT_{2A} serotonin receptor antagonist selected from: altanserin, amitriptyline, amoxapine, benperidol, (+)butaclamol, d-butaclamol, chlorpromazine, chlorprothixene, cinanserin, clopipazan, clozapine, cyamemazine, cyproheptadine, droperidol, ergotamine, alpha-flupenthixol, fluphenazine, fluspiperone, iloperidone, isoclozapine, ketanserin, lisuride, loxapine, lurasidone, mesulergine, metergoline, methergine, methiothepin, methylergonovine, methysergide, metitepin, mianserin, mirtazapine, octoclothepin, olanzapine, ORG-5222 asenapine, perospirone, pipamperone, pirenperone, prazosin, propericiazine, rilapine, risperidone, ritanserin, RMI 81,582, sertindole, setoperone, spiperone, N-Me-spiperone, thioridazine, cis-thiothixene, tiosperone, xylamidine, ziprasidone, and zotepine.

In some embodiments, the present disclosure provides methods comprising: administering to a patient diagnosed with Dravet syndrome or Lennox-Gastaut syndrome a liquid formulation comprising a pharmaceutically acceptable carrier and a therapeutically effective amount of fenfluramine or a pharmaceutically acceptable salt thereof; and advising the patient by a label, a package insert or a medication guide accompanying the formulation to avoid treatment with a serotonin receptor antagonist.

In some embodiments, a serotonin receptor antagonist is cyproheptadine. In some embodiments, a serotonin receptor antagonist is a 5-HT_{1D} serotonin receptor antagonist or a 5-HT_{2A} serotonin receptor antagonist.

In some embodiments, a serotonin receptor antagonist is a 5-HT_{1A} serotonin receptor antagonist and/or 5-HT_{2C} serotonin receptor antagonist that is selected from cyproheptadine, clozapine, doxepin, quetiapine, ketotifen, pizotifen, perphenazine, mianserin, mirtazapine, risperidone and asenapine.

In some embodiments, fenfluramine is administered or has been administered in a dose that is in a range of from 10.0 mg/kg/day to 0.01 mg/kg/day. In certain embodiments, a therapeutically effective dose of fenfluramine is within a range of about 0.1 mg/kg/day to about 2.5 mg/kg/day.

In some embodiments, fenfluramine is administered or has been administered in a dosage form selected from the group consisting of oral, injectable, transdermal, inhaled, nasal, buccal, rectal, vaginal and parenteral delivery.

In some certain embodiments, fenfluramine is administered or has been administered as an oral solution. In some certain embodiments, fenfluramine is administered or has been administered in an amount within a range of 10 mg to 200 mg. In some certain embodiments, fenfluramine is administered or has been administered in an amount that is 120 mg or less, 60 mg or less, 30 mg or less, and 20 mg or less.

In some embodiments, a therapeutically effective total daily dose of fenfluramine is no more than 40 mg, no more than 30 mg, or no more than 20 mg.

In some embodiments, the present disclosure provides kits comprising: a container comprising a fenfluramine formulation, and a package insert, a label or a medication guide comprising content warning against co-administration with a serotonin (5-HT)-receptor antagonist.

In some embodiments, a serotonin receptor antagonist is cyproheptadine. In some embodiments, a serotonin receptor antagonist is an antagonist at one or more of subtypes chosen from 5-HT_{1A}, 5-HT_{1D}, 5-HT_{2A} and 5-HT_{2C}.

In some certain embodiments, a serotonin receptor antagonist is a 5-HT_{1D} serotonin receptor antagonist or a 5-HT_{2A} serotonin receptor antagonist.

In some embodiments, a serotonin receptor antagonist is a 5-HT_{1A} serotonin receptor antagonist and/or 5-HT_{2C} serotonin receptor antagonist that is selected from cyproheptadine, clozapine, doxepin, quetiapine, ketotifen, pizotifen, perphenazine, mianserin, mirtazapine, risperidone and asenapine.

In some embodiments, fenfluramine is formulated for oral administration, as an injectable, transdermal administration, nasal administration, buccal administration, rectal administration, vaginal administration, and/or parenteral administration.

In some embodiments, fenfluramine is provided as a liquid formulation.

In some embodiments, a kit comprises a package insert, a label or a medication guide further informs that (a) fenfluramine can be used to treat Dravet syndrome or Lennox-Gastaut syndrome, and/or (b) fenfluramine treatment may result in weight loss, wasting, and/or loss of appetite.

In some embodiments, the present disclosure provides kits comprising: a container comprising a plurality of doses of a formulation comprising a pharmaceutically acceptable carrier and an active ingredient comprising fenfluramine; instructions associated with the container for treating the patient diagnosed with epilepsy or epileptic encephalopathy wherein the instructions include administering the formulation to the patient if the patient while not administering a serotonin receptor antagonist.

In some embodiments, the present disclosure provides kits comprising: an oral solution comprising 2.5 milligram of fenfluramine in each milliliter of liquid solution; and instructions that indicate (i) dosing the patient based on patient weight and volume of oral solution administered and (ii) to avoid co-treatment of the patient with a serotonin receptor antagonist.

In some embodiments, the present disclosure provides kits comprising: a solid oral formulation of fenfluramine; and instructions that indicate to avoid co-treatment of the patient with a serotonin receptor antagonist. In some embodiments, a solid oral formulation is selected from the group consisting of: a tablet, a disintegrating table, a capsule, a lozenge, and a sachet.

In some embodiments, the present disclosure provides kits comprising: a formulation of fenfluramine as a transdermal patch; and instructions that indicate to avoid co-treatment of the patient with a serotonin receptor antagonist.

According to one aspect of the present disclosure, herein provided is a method of treating, and/or preventing a symptom of epilepsy or epileptic encephalopathy by administering a therapeutically effective dose of fenfluramine or a pharmaceutically acceptable salt thereof to the patient, wherein the patient is not being treated with a potent serotonin receptor antagonist.

According to another aspect, herein provided is a kit, comprising a fenfluramine formulation, a package, and a package insert comprising content informing a physician or caregiver to monitor patients when a potent serotonin (5-HT)-receptor antagonist is administered with fenfluramine. In some embodiments, the 5-HT receptor antagonist is a potent 5-HT_{2A} receptor antagonist. In some embodiments, the 5-HT receptor antagonist is a potent 5-HT_{2C} receptor antagonist. In some embodiments, the 5-HT receptor antagonist is a potent 5-HT_{1A} receptor antagonist. In some embodiments, the 5-HT receptor antagonist is a potent 5-HT_{1D} receptor antagonist. In some embodiments the 5-HT receptor antagonist is a potent 5-HT_{2A} and 5-HT_{2C} antagonist. In some embodiments the 5-HT receptor antagonist is a potent 5-HT_{1A} and 5-HT_{1D} antagonist. In some embodiments the potent 5-HT receptor antagonist is cyproheptadine. In some embodiments the potent 5-HT receptor antagonist is both a 5-HT_{2A} and 5-HT_{1D} antagonist. In some embodiments, the monitoring is to detect an increase in frequency or severity of seizures in the patient. In some embodiments, a potent 5-HT receptor antagonist has a Ki that is ≤1000 nM (e.g., a Ki that is ≤500 nM, a Ki that is ≤250 nM, a Ki that is ≤100 nM, a Ki that is ≤50 nM, a Ki that is ≤25 nM, or a Ki that is ≤10 nM). In some embodiments, a potent 5-HT receptor antagonist exhibits serotonergic blocking activity at one or more of the 5-HT_{1A}, 5-HT_{1D}, 5-HT_{2A} and 5-HT_{2C} receptors at the plasma concentration range associated with its efficacy for the condition being treated.

According to another aspect, herein provided is a kit, comprising a fenfluramine formulation, a package, and a package insert comprising content informing a physician or caregiver to monitor patients when a serotonin (5-HT)-receptor antagonist is administered with fenfluramine. In some embodiments, the 5-HT receptor antagonist is a 5-HT_{2A} receptor antagonist. In some embodiments, the 5-HT receptor antagonist is a 5-HT_{2C} receptor antagonist.

In some embodiments the potent 5-HT receptor antagonist is cyproheptadine. In some embodiments, the 5-HT receptor antagonist is a 5-HT_{1A} receptor antagonist. In some embodiments, the 5-HT receptor antagonist is a 5-HT_{1D} receptor antagonist. In some embodiments the 5-HT receptor antagonist is a 5-HT_{2A} antagonist and a 5-HT_{2C} antagonist. In some embodiments the 5-HT receptor antagonist is a 5-HT_{1A} antagonist and a 5-HT_{1D} antagonist. In some embodiments the 5-HT receptor antagonist is a 5-HT_{2A} antagonist and a 5-HT_{1D} antagonist. In some embodiments, the monitoring is to detect an increase in seizures in the patient.

According to another aspect, herein provided is a kit, comprising a fenfluramine formulation, a package, and a package insert comprising content informing a physician or caregiver to avoid co-administering one or more potent serotonin (5-HT)-receptor antagonists with fenfluramine to a patient. In some embodiments, the potent 5-HT receptor antagonist is a 5-HT_{2A} receptor antagonist. In some embodiments, the potent 5-HT receptor antagonist is a 5-HT_{2C} receptor antagonist. In some embodiments the potent 5-HT receptor antagonist is cyproheptadine. In some embodiments, the potent 5-HT receptor antagonist is a 5-HT_{1A} receptor antagonist. In some embodiments, the potent 5-HT receptor antagonist is a 5-HT_{1D} receptor antagonist. In some embodiments the potent 5-HT receptor antagonist is a 5-HT_{2A} antagonist and a 5-HT_{2C} antagonist. In some embodiments the potent 5-HT receptor antagonist is a 5-HT_{1A} antagonist and a 5-HT_{1D} antagonist. In some embodiments the potent 5-HT receptor antagonist is a 5-HT_{2A} antagonist and a 5-HT_{1D} antagonist.

According to another aspect, herein provided is a kit, comprising a fenfluramine formulation, a package, and a package insert comprising content informing a physician or caregiver that co-administering one or more potent serotonin (5-HT)-receptor antagonists with fenfluramine to an epileptic patient may decrease the anti-seizure efficacy of fenfluramine. In some embodiments, the potent 5-HT receptor antagonist is a 5-HT_{2A} receptor antagonist. In some embodiments, the potent 5-HT receptor antagonist is a 5-HT_{2C} receptor antagonist. In some embodiments the potent 5-HT receptor antagonist is cyproheptadine. In some embodiments, the potent 5-HT receptor antagonist is a 5-HT_{1A} receptor antagonist. In some embodiments, the potent 5-HT receptor antagonist is a 5-HT_{1D} receptor antagonist. In some embodiments, the potent 5-HT receptor antagonist is a 5-HT_{2A} antagonist and a 5-HT_{2C} antagonist. In some embodiments, the potent 5-HT receptor antagonist is a 5-HT_{1A} antagonist and a 5-HT_{1D} antagonist. In some embodiments, the potent 5-HT receptor antagonist is a 5-HT_{2A} antagonist and a 5-HT_{1D} antagonist. In some embodiments, the anti-seizure efficacy is decreased. In some embodiments, the anti-seizure efficacy is abolished.

According to another aspect, herein provided is a kit, including a container having a plurality of doses of a formulation comprising a pharmaceutically acceptable carrier and an active ingredient comprising fenfluramine; and instructions for treating a patient diagnosed with epilepsy or epileptic encephalopathy wherein the instructions informing a physician or caregiver that anti-serotonergic agents should not be administered to patients receiving fenfluramine. In some embodiments, the 5-HT receptor antagonist is a 5-HT_{2C} receptor antagonist. In some embodiments, the 5-HT receptor antagonist is a 5-HT_{2A} receptor antagonist. In some embodiments, the 5-HT receptor antagonist is a 5-HT_{1A} receptor antagonist. In some embodiments, the 5-HT receptor antagonist is a 5-HT_{1D} receptor antagonist.

According to another aspect, herein provided is a kit, comprising a first container having a plurality of doses of a formulation including a pharmaceutically acceptable carrier and an active ingredient comprising fenfluramine; and instructions for treating a patient diagnosed with epilepsy or epileptic encephalopathy wherein the instructions inform a physician or caregiver that co-administration of one or more histamines chosen from the group of cyproheptadine; ketotifen; pizotifen, epinastine; and desloratadine may decrease the efficacy of fenfluramine in the patient. In some embodiments, the antihistamine is cyproheptadine.

According to another aspect, a method is provided for treating a symptom of epilepsy or epileptic encephalopathy in a patient diagnosed with epilepsy or epileptic encephalopathy, comprising administering a therapeutically effective dose of fenfluramine or a pharmaceutically acceptable salt thereof to the patient, wherein the patient is advised by a product insert, a medication guide or a label accompanying the fenfluramine to avoid treatment with potent serotonin receptor antagonists. In some embodiments, the 5-HT receptor antagonist is a 5-HT_{2C} receptor antagonist. In some embodiments, the 5-HT receptor antagonist is a 5-HT_{2A} receptor antagonist. In some embodiments, the 5-HT receptor antagonist is a 5-HT_{1A} receptor antagonist. In some embodiments, the 5-HT receptor antagonist is a 5-HT_{1D} receptor antagonist.

According to another aspect, a method is provided for treating a patient diagnosed with (a) Dravet syndrome and/or Lennox-Gastaut syndrome, and (b) extreme weight loss, wasting, cachexia and/or loss of appetite, by administering to the patient a liquid formulation comprising a pharmaceutically acceptable carrier and a therapeutically effective amount of fenfluramine or a pharmaceutically acceptable salt thereof; and advising the patient by a product insert, a medication guide or a label accompanying the formulation to avoid treatment with potent serotonin receptor inhibitors.

According to another aspect, the present disclosure provides a method of increasing patient safety by informing a prescribing physician who provides fenfluramine to a patients that serotonin receptor inhibitors, particularly potent inhibitors at 5-HT_{1A}, 5-HT_{1D}, 5-HT_{2A} and 5-HT_{2C}, may cause a loss of anti-seizure effect and should not be co-prescribed to the patient.

Throughout the description, where systems or compositions are described as having, including, or comprising specific components, or where methods are described as having, including, or comprising specific steps, it is contemplated that, additionally, there are systems or compositions of the present invention that consist essentially of, or consist of, the recited components, and that there are methods according to the present invention that consist essentially of, or consist of, the recited steps.

It should be understood that the order of steps or order for performing certain action is immaterial so long as the invention remains operable. Moreover, two or more steps or actions may be conducted simultaneously.

These and other objects, advantages, and features of the invention will become apparent to those persons skilled in the art upon reading the details of the methods of treating symptoms of epilepsy or epileptic encephalopathy as more fully described below.

### IV. BRIEF DESCRIPTION OF THE FIGURES

The Drawing included herein, which is composed of the following Figures, is for illustration purposes only and not for limitation.

FIG. 1 is a table showing receptor binding affinities for a variety of antipsychotic drugs that may be used to treat conditions other that psychosis or schizophrenia.

FIG. 2 is a table of Ki of 5-HT receptor antagonists on different 5-HT receptors, with the corresponding effect on fenfluramine anti-seizure activity in a IVIES model. Shading in each cell represents an average Ki value observed. Solid black shading represents a Ki of ≤1 nM; shading with horizontal lines represents a Ki of >1 and ≤10 nM; shading with vertical lines represents a Ki of >10 and ≤100 nM; shading with vertical cross-hatched lines represents a Ki of >100 and ≤1000 nM, shading with angled cross-hatched represents a Ki of >1000 and ≤10000 nM, and shading with dots represents a K1 of >10000 nM. Outliers were removed, which were defined as values more than 2 standard deviations from the mean. The last column provides a summary of the number of animals protected in an MES model, as described in the Examples.

FIG. 3 provides a summary table of FFA anti-seizure activity in combination with various 5-HT antagonists in a zebrafish model. *** indicates highest potency, with decreasing potency with ** and *, respectively.

### V. CERTAIN DEFINITIONS

In order for the present invention to be more readily understood, certain terms are first defined below. Additional definitions for the following terms and other terms are set forth throughout the specification.

It is noted that as used herein and in the appended claims, the singular forms “a”, “an”, and “the” include plural referents unless the context clearly dictates otherwise. Thus, for example, reference to “a seizure” includes a plurality of such seizures and reference to “the drug” includes reference to one or more drugs and equivalents thereof known to those skilled in the art, and so forth.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse-affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing amelioration and/or regression of the disease. With respect to epilepsy or epileptic encephalopathy, symptoms that may be improved by treatment include occurrence, frequency or duration of seizures, for example.

A “therapeutically effective amount” or “efficacious amount” refers to the amount of a compound or agent that, when administered to a mammal or other subject for treating a disease, is sufficient to effect such treatment for the disease. The “therapeutically effective amount” will vary depending on the compound or the agent, the disease and its severity and the age, weight, etc., of the subject to be treated.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

To avoid doubt, the term “prevention” of seizures means the total or partial prevention (inhibition) of seizures. Ideally, the methods of the present invention result in a total prevention of seizures. However, the invention also encompasses methods in which the instances of seizures are decreased in frequency by at least 40%, at least 50%, at least 55%, at least 60%, at least 65%, at least 70%, at least 75%, at least 80%, at least 85%, or at least 90%. In addition, the invention also encompasses methods in which the instances of seizures are decreased in duration or severity by at least 40%, at least 50%, at least 55%, at least 60%, at least 65%, at least 70%, at least 75%, at least 80%, at least 85%, or at least 90%.

Before the present method, kits and formulations are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. The publications discussed herein are provided solely for their disclosure prior to the filing date of the present application. All publications mentioned herein are incorporated herein by reference to disclose and describe the methods and/or materials in connection with which the publications are cited. Nothing herein is to be construed as an admission that the present invention is not entitled to antedate such publication. Further, the dates of publication provided may be different from the actual publication dates which may need to be independently confirmed. It is understood that the present disclosure supersedes any disclosure of an incorporated publication to the extent there is a contradiction.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### VI. DETAILED DESCRIPTION OF THE INVENTION

### Fenfluramine

Fenfluramine, i.e. 3-trifluoromethyl-N-ethylamphetamine is an amphetamine derivative having the structure:

[chemical structure]

### Systematic (IUPAC) Name (RS)—N-ethyl-1-[3-(trifluoromethyl)phenyl]propan-2-amine

Fenfluramine was first marketed in the US in 1973 to treat obesity. However, in 1997, it was withdrawn from the US and global market as its use was associated with the onset of cardiac valvulopathy and pulmonary hypertension.

Without being bound by theory, the adverse effects associated with the use of fenfluramine as an anorexic agent are thought to be attributable to the interaction of fenfluramine's major metabolite norfenfluramine with the 5-HT₂B receptor, which is associated with heart valve fibrosis and hypertrophy. Fenfluramine is metabolized in vivo into norfenfluramine by cytochrome P450 enzymes in the liver. Cytochrome P450 enzymes such as CYP2D6, CYP2B6 and CYP1A2 are primarily responsible for the production of norfenfluramine from fenfluramine in humans. The enzymes CYP2C9, CYP2C19 and CYP3A4 are also involved. Such metabolism includes cleavage of an N-ethyl group to produce norfenfluramine as shown below.

[chemical structure]

Fenfluramine acts primarily as a serotonin releasing agent. Fenfluramine and its major metabolite, norfenfluramine, were reported to be potent substrates for norepinephrine transporters. (Rothman, et al., J. Pharmacol. Exp. Ther. 305(3):1191-9). Fenfluramine causes the release of serotonin by disrupting vesicular storage of the neurotransmitter, and reversing serotonin transporter function. Fenfluramine also acts as a norepinephrine releasing agent to a lesser extent, particularly via its active metabolite norfenfluramine. In addition to monoamine release, while fenfluramine binds only very weakly to the serotonin 5-HT₂ receptors, norfenfluramine binds to and activates the serotonin 5-HT_{2B} and 5-HT_{2C} receptors with high affinity and the serotonin 5-HT_{2A} receptor with moderate affinity. The result of the increased serotonergic and noradrenergic neurotransmission is a feeling of fullness and reduced appetite. Thus, in subjects treated with fenfluramine, weight loss, anorexia and/or wasting may be observed.

Despite past cardiovascular safety concerns that arose when high doses of fenfluramine were used for treatment of adult obesity, attempts have been made to identify further therapeutic uses for that product, while weighing the known cardiovascular risks of fenfluramine against potential therapeutic benefits.

The present disclosure encompasses a recognition that fenfluramine is highly efficacious for the treatment of seizures in clinical studies on the treatment of certain forms of epilepsy. As is the subject of several related US patent applications (US 2017-0056344-A1; US 2017-0071949-A1; US 2018-0055789-A1; and US 2018-0092864-A1) and issued US patents (U.S. Pat. Nos. 9,549,909; 9,610,260; 9,603,814; and 9,603,815), fenfluramine was found to be useful in treating, ameliorating, or minimizing the symptoms of epilepsies or epileptic encephalopathies, thus reducing the number, intensity and/or length of seizures. Fenfluramine is particularly useful in the treatment of epilepsies, and in particular, epileptic encephalopathies such as Dravet syndrome and Lennox-Gastaut syndrome. Provided herein are improved methods of treating and/or ameliorating epilepsy (such as, e.g., Dravet syndrome) that include treatment of patients with fenfluramine (e.g., administering fenfluramine and/or include patients to whom fenfluramine has been or will be administered).

In some embodiments, epilepsy patient(s) whose seizures are being treated with fenfluramine, may be characterized by weight loss, anorexia and/or wasting. In some embodiments, patient(s) whose seizures are being treated with fenfluramine may be co-administered an appetite stimulant.

### Epilepsy

Disorders for which new treatment options are sorely needed include epilepsy or epileptic encephalopathy, and in particular, epilepsy syndromes which are refractory to known treatments. Epilepsy is a functional disturbance of the central nervous system (CNS) induced by abnormal electrical discharges and marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

In some embodiments, epilepsy and/or epileptic encephalopathy in the context of the present disclosure is or comprises Dravet syndrome, Lennox Gastaut syndrome, Rett syndrome, Doose syndrome, West syndrome, Tuberous Sclerosis Complex (TSC), Dup15q Syndrome, CDKL5 Deficiency Disorder, epileptic encephalopathy associated with mutation(s) in the PCDH19 Gene, and/or an epileptic encephalopathies associated with mutations in sodium channel genes. In some embodiments, epilepsy and/or epileptic encephalopathy in the context of the present disclosure is or comprises Dravet syndrome, Lennox Gastaut syndrome, Rett syndrome, Doose syndrome, and/or West syndrome.

A large number of compounds may be used to treat different types of epilepsy or epileptic encephalopathy, and different epilepsy subtypes respond differently to different anticonvulsant drugs. For example, cannabidiol has been studied for treatment of drug-resistant seizures in Dravet syndrome and was reported to reduce convulsive-seizure frequency (Devinsky, et al., 2017, New Engl. J. Med. 376(21):2011-2020). The precise mechanisms by which EPIDIOLEX exerts its anticonvulsant effect in humans are unknown. Cannabidiol does not appear to exert its anticonvulsant effects through interaction with cannabinoid receptors. (Epidiolex Highlights of Prescribing Information, § 12.1).

However, while a particular drug may be effective against one form of epilepsy, it may be wholly ineffective against others, or even contra-indicated due to exacerbation of symptoms, such as worsening the frequency and severity of the seizures. For instance, seizures in Dravet syndrome occur principally as a result of mutations in a sodium channel (Nav1) and sodium channel blockers including carbamazepine, oxcarbazepine, lamotrigine, lacosamide, rufinamide, phenytoin, and fosphenytoin are contra-indicated in Dravet syndrome as these drugs are known to lead to a greater incidence of seizures in almost all Dravet syndrome patients through clinical experience. Whether or not a particular drug is preferred with respect to a particular type of epilepsy is based largely on clinical experience as well as controlled clinical trials when available.

### Epilepsy-Associated Co-Morbidities

In addition to seizures, particularly in the epileptic encephalopathies, there a number of central nervous system mediated co-morbidities associated with these epilepsies which include: behavioral problems, including autism spectrum symptoms, such as obsessive-compulsive behaviors, hyperactivity; social withdrawal, aggressive or combative behaviors, cognitive difficulties, problems with sleep, anxiety, ataxias, and respiratory difficulties (some being autonomic nervous system related, particularly in Rett syndrome). Other common co-morbidities present in many epileptic syndromes include problems with depression, anxiety and sleep. Physicians try to manage these co-morbidities as well as seizures as they contribute to lower quality of life for both the patients and their caregivers.

Additionally, anti-epileptic drugs (AEDs) used to treat seizures in epileptic syndromes may themselves cause side effects, for instance, some AEDs are associated with weight loss and cognitive difficulties. The known anorectic effects of fenfluramine are associated with loss of appetite, weight loss and/or wasting. In clinical studies in epilepsy patients using low-dose fenfluramine, such as less than 30 mg/day of fenfluramine hydrochloride, some patients did experience weight loss, in some cases weight was regained after an initial period of weight loss and in others, weight loss persisted. Cognitive side effects include problems with thinking, remembering, sustained attention, concentrating or word finding difficulties. Cognitive difficulties are most likely to occur when two or more AEDs are used together (polytherapy). Polytherapy is common in the refractory epilepsies.

There are a number of agents that may be used in treatment of the above described co-morbid conditions or AED side effects. For instance, there a number of agents that might be considered for appetite stimulation in epileptic patients are first generation H1 antagonists since they penetrate into the CNS. Additionally, centrally acting H1 antagonists are known to produce drowsiness and sedation. Thus for patients experiencing weight loss, or failure to gain weight during childhood, or for patients with sleep disturbances, or particularly for those experiencing both a CNS penetrant H1 antagonist, would appear to manage these symptoms.

In some embodiments, subjects with epilepsy and/or epileptic encephalopathies are also characterized by one or more characteristics of autism spectrum symptoms, such as, for example, obsessive-compulsive behaviors. Obsessive compulsive behaviors that include stereotypy, repetitive behavior and compulsions are observed in some epileptic patients, including Rett patients.

In some embodiments, subjects with epilepsy and/or epileptic encephalopathy characterized by one or more autism spectrum symptoms, can be treated with medications that are selective serotonin re-uptake inhibitors, clomipramine (Anafranil) first line treatment for patients 10 years and older; fluoxetine (Prozac) for patients 7 years and older; fluvoxamine for patients 8 years and older; paroxetine (Paxil, Pexeva) for adults and sertraline (Zoloft) for patients 6 years and older. Clomipramine is used off-label for OCD and has been found more effective than fluvoxamine, and other SSRIs [Greist, J H (January 1995). Archives of General Psychiatry, 52 (1): 53-60.]

Other agents used in autism spectrum symptoms include typical and atypical antipsychotics. Typical antipsychotics are first generation medications that include phenothiazine derivatives such as chlorpromazine and butyrophenone derivatives which include drugs whose generic name have the ending stems “-peridol”, such as haloperidol and droperidol and those ending with “-perone”, such as spiperone and piamperone, among other structural classes. These first generation drugs are less favored today owing to side effects. Atypical antipsychotics are preferred in medicine today since they lack or exhibit fewer side effects, such as extrapyramidal side effects. Atypicality is often defined in a drug compound as having dopamine D2 antagonism and 5-HT_{2A} antagonism. 5-HT_{2A} antagonism is able to stimulate dopamine release in certain areas of the brain. 5-HT_{2A} antagonism causes dopamine release in certain brain areas, including the striatum and nigrostriatal pathway, and this pharmacological action hypothetically explains the atypical clinical properties of these agents that distinguish them from conventional antipsychotics, namely low EPS and efficacy for negative symptoms. [See, for example, Stahl. S, “Antipsychotics and Mood Stabilizers: Stahl's Essential Psychopharmacology”, 3^{rd} Edition, Cambridge University Press].

In some embodiments, subjects with epilepsy and/or epileptic encephalopathies are also characterized by a sleep disorder. Sleep disorders, including sleep induction and sleep maintenance are treated in the short term with sedatives of the benzodiazepine class and “Z-drug” (zolpidem, zopiclone, zaleplon) hypnotics all of which act as GABA_{A} receptor agonists. These drugs are generally not useful in patients who have chronic sleep problems as a co-morbidity of an epilepsy syndrome. Potent centrally acting H1 histamine antagonists are associated with sedation and sleep. Second generation anti-histamines were designed to lack central nervous system penetration, either though protonation at physiologic pH, being substrates for efflux mechanism in the blood brain barrier or other structural modifications affecting lipophilicity of the molecule. Second generation antihistamines then have peripheral effects, such as in the gastrointestinal tract, large blood vessels, skin, eyes, mucous membranes and bronchial smooth muscle. First generation antihistamines can enter the CNS and so also antagonize histamine binding to histaminergic neuron receptors. Central histamine receptors are associated with modulating the circadian cycle (i.e., cycle of sleeping and wakefulness). Another effect of central histamine antagonism is associated with appetite and weight gain. In fact, one study demonstrated a direct correlation between the H1 antagonist activity with weight gain in a group of antipsychotic drugs [Kroeze, W. et al., Neuropsychopharmacology (2003) 28, 519-526]. One group has implicated inhibition of histamine H1 receptor function in seizure induction. [Swiader, M. (2004) 14(4):307-18.] Other investigators have hypothesized that proconvulsant effects of clozapine are mediated by H1 antagonist effects per se and though increasing endogenous leptin levels. [Ghanizadeh, A., Psychiatria Danubina, 2010; Vol. 22, No. 4, pp 552-553] Other groups have found a lack of proconvulsant activity depending on the specific antihistamine, dosing level and animal model used.

In children ages 1 through 12 years, chlorpromazine is used to treat severe behavioral problems (such as combative or explosive behavior) or hyperactivity with excessive motor activity.

Loxapine is used as a tranquilizer for which the exact mode of action has not been established, however, it is believed that by antagonizing dopamine and serotonin receptors, there is a marked cortical inhibition which can manifest as tranquilization and suppression of aggression.

Doxepin (Silenor®) is a tricyclic antidepressant that has been repurposed and FDA approved (2010) as a low dose agent (3 to 6 mg/day) for insomnia characterized by difficulty maintaining sleep. The primary mechanism of action is described as H1 antagonism for sleep maintenance.

Many of the drugs discussed herein have some common structural features as shown in Table I and which are shared with a number of other drugs including drugs marketed as antihistamines, hypnotics, tranquilizers, antidepressants, antipsychotics and anticholinergics:

[chemical structure]

TABLE 1
Drugs with a Structure based on Structure 2

| Drug | X—A | | Y—Z | |
|---|---|---|---|---|
| (Uses) | (divalent atom “X” is shown at left) | R1 | (atom “Y” is at left) | Q |
| Doxepin | C═C—(CH₂)₂—NMe₂ | H | —CH₂—O— | CH |
| Silenor ® | | | | |
| (Depression, | | | | |
| sleep) | | | | |
| Loxapine Loxitane ® Tranquilier, antipsychotic | —O— | Cl | [chemical structure] | CH |
| Clomipramine | —N—(CH₂)₃—NMe₂ | Cl | —CH₂—CH₂— | CH |
| Depression, | | | | |
| OCD, | | | | |
| symtoms of | | | | |
| autism | | | | |
| Mianserin Tolvon ® (Depression, sleep) | —CH2— | | [chemical structure] | CH |
| Mirtazapine Remeron ® (Depression, sleep, appetite) | —CH2— | | [chemical structure] | N |
| Clozapine Clozaril ® Schizophrenia | —NH— | Cl | [chemical structure] | CH |
| Perphenazine Trilafon ® Adjunctive therapy for agitated depression | [chemical structure] | Cl | —S— | CH |
| Asenapine Saphris ® (Psychosis, bi-polar disorder) | —O— | Cl | [chemical structure] | CH |
| Cyproheptadine (Allergies, increasing appetite) | [chemical structure] | H | —CH═CH— | CH |

Carbamazepine, noted as a sodium channel blocking antiepileptic drug, also has the tricycling ring structure of
Structure 2 where Y—Z is —HC═CH—, X—A is N—C(O)—NH2, Q is carbon and R1 is hydrogen (see Table 4,
Compound #7). Carbamazepine is not a serotonergic antagonist and has been reported to be a weak serotonin
releaser Dailey, et al, Epilepsia 1998 Oct;39(10):1054-63.

For example, in children ages 1 through 12 years, chlorpromazine is used to treat severe behavioral problems (such as combative or explosive behavior) or hyperactivity with excessive motor activity.

Loxapine, a dibenzoxazepine compound, represents a subclass of tricyclic antipsychotic agents, chemically distinct from the thioxanthenes, butyrophenones, and phenothiazines. Loxapine is used as a tranquilizer for which the exact mode of action has not been established, however, it is believed that by antagonizing dopamine and serotonin receptors, there is a marked cortical inhibition which can manifest as tranquilization and suppression of aggression.

Cyproheptadine (C₂₁H₂₁N) is in the family of drugs called antihistamines, and is typically used orally to treat asthma, allergies and colds, and to relieve itching caused by hives and other skin disorders. Structures related to cyproheptadine are loratadine; desloratadine, azatadine, ketotifen and pizotifen. One of the side effects of cyproheptadine is increased appetite and weight gain, For example, the effect of cyproheptadine on water and food intake and on body weight has been studied in fasted adult and weanling rats; a reduction of serotonin system activity was observed to increase food intake. (Ghosh M N, Parvathy S. Br. J. Pharmacol. 1973, 48(2):328P-329P). The appetite stimulating effect of cyproheptadine has led to its off-label use to stimulate weight gain in patients for treatment of symptoms of wasting and/or anorexia, as well as in people with cystic fibrosis and in patients exhibiting weight loss and/or cachexia caused by cancer and its treatment.

In addition to its antihistamine action, cyproheptadine also competes with serotonin for binding at other receptor sites, and has anticholinergic, antiserotonergic, antidopaminergic, local anesthetic and sedative properties. Many of the drugs that share common features with the drugs listed in Table I have complex pharmacological profiles with varying degrees of antagonist activity based on subtle structural differences. For example, cyclobenzaprine is prescribed as a muscle relaxant while amitriptyline is administered for depression.

Structure 3 (cyclobenzaprine) and Structure 4 (amitryptiline):

[chemical structure]

There is a long-felt need to provide an improved method for treating or preventing and/or ameliorating seizures experienced by sufferers of epilepsy or epileptic encephalopathy, particularly in children and young adults, e.g., Dravet syndrome and/or Lennox-Gastaut syndrome. However, as is described hereinbelow, in some forms of epilepsy, patients who are being administered fenfluramine to treat symptoms of epilepsy or epileptic encephalopathy (e.g., seizures) also may exhibit co-morbid psychiatric conditions, weight loss, anorexia and/or wasting, which a treating physician would consider administering other agents. Thus, as part of a treatment regimen, physicians may consider prescribing certain CNS penetrant drugs for co-administration with fenfluramine for treatment of co-morbid psychiatric conditions or weight loss in epileptic patients. The present disclosure relates to the discovery that certain serotonin receptor antagonists are contraindicated in the treatment of symptoms of epilepsy or epileptic encephalopathy using fenfluramine.

### Fenfluramine as an Anti-Epileptic

Fenfluramine has therapeutic use in the treatment of certain epilepsies and/or epileptic encephalopathies, including, but not limited to, Dravet syndrome (see for example U.S. Pat. No. 9,549,909), Lennox Gastaut syndrome (U.S. Patent Publication No. 20170056344), Rett syndrome, Doose syndrome and West syndrome. The mechanism of action of fenfluramine in treatment of epileptic encephalopathies is not completely understood and may involve several pathways in producing its anti-epileptic effects. As described above, the fenfluramine metabolite, norfenfluramine, binds to and activates the serotonin 5-HT_{2B} and 5-HT_{2C} receptors with high affinity and the serotonin 5-HT_{2A} receptor with moderate affinity. Fenfluramine has less affinity and is less potent as a direct agonist at serotonin receptors (Rothman, R B, et al., Circulation, 2000, 102(23):2836-2841). However, fenfluramine does act as a serotonin releaser via its effects on the serotonin transporter and is thought to produce some of its effects via indirect agonism of certain 5-HT receptors via serotonin release in neuronal synapses, such as via 5-HT_{1A} and 5-HT_{1D}, 5-HT_{2A} and 5-HT_{2C} receptors as demonstrated in a zebrafish model of Dravet syndrome, (Sourbron, J., et al, Front. Pharmacol. 2017; 8:191).

Fenfluramine is a racemic mixture of two enantiomers, dexfenfluramine and levofenfluramine, and has been reported to increase the levels of serotonin, a neurotransmitter that regulates mood, appetite and other functions. Fenfluramine is classified as a serotonin releasing drug that also has some effect on reduction of serotonin reuptake, although it does not have a typical 5-HT reuptake inhibition mechanism.

In addition to its effects on serotonin, fenfluramine has been demonstrated to be a sigma-1 receptor positive allosteric modulator. Sigma-1 allosteric agonism has been shown in several animal models of epilepsy to reduce or suppress seizures. Although the mechanism of this anti-seizure effects is still being investigated, one study demonstrated that sigma-1 agonists have an indirect effect on IK (inward potassium currents) through Kv2.1, a-potassium channel subunit which is expressed at high levels in most mammalian CNS neurons and plays a crucial role in regulating neuronal excitability. Native neuronal IK channels are mainly composed of members of the Kv2 subfamily [He, Yan-Lin, et al., PLoS ONE, 2012, vol. 7, issue 7, p. e41303].

That the serotonergic activity of fenfluramine is not the sole mechanism of action is illustrated by the existence of dozens and dozens of serotonergic of agents known in medicine and the scientific literature, none of which none have regulatory approval as an anti-epileptic agent and are not commonly used “off-label” in clinical practice to treat seizures in most types of epilepsy.

Serotonin (also known as “5-hydroxytryptamine” or “5-HT”) is a monoaminergic neurotransmitter believed to modulate numerous sensory, motor and behavioral processes in the mammalian nervous system. These diverse responses are elicited through the activation of a large family of receptor subtypes. The complexity of the serotonin signaling system and the paucity of selective drugs have made it difficult to define specific roles for 5-HT receptor subtypes, or to determine how serotonergic drugs modulate mood and behavior. Of the many subtypes of serotonin receptors, the 1B and 2C subtypes are most strongly implicated in modulating feeding and body weight, and these receptors are expressed in hypothalamic regions believed to be involved in food intake regulation. Numerous drugs are known to affect serotonin levels in the CNS and they man function as 5-HT reuptake inhibitors, releasers, catabolism inhibitors, or precursor molecules in its biosynthesis. Serotonin syndrome, a potentially fatal result of too much serotonin in the CNS, is associated with some serotonergic drugs and particularly when two serotonergic with differing mechanisms are combined. Fenfluramine should not be combined with other drugs that enhance serotonin, some examples of incompatible drugs are listed in Table II below.

TABLE 2
Drugs Associated with Serotonin Syndrome

| | |
|---|---|
| Amitriptyline (e.g., Elavil) | Meperidine (e.g., Demerol) |
| Amphetamines (e.g., Adderall) | Moclobemide |
| Clomipramine (e.g., Clomicalm) | Paroxetine (e.g., Paxil) |
| Venlafaxine (e.g., Effexor) | Lithium |
| Hydroxytryptophan | MAOIs: e.g. Selegiline (e.g., Anipryl); |
| | Tranylcypromine; Phenelzine |
| Imipramine (Tofranil) | Sertraline (e.g., Zoloft) |
| Isocarboxazid | Tryptophan |

Both 1B and 2C receptor agonists have been found to suppress feeding in rodents, and 2C receptor knockout mice display chronic hyperphagia and obesity. Furthermore, knockout mice lacking functional 5-HT₂C receptors (previously termed 5-HT_{1C}) were found to be hyperphagic, which led to obesity, partial leptin resistance, increased adipose deposition, insulin resistance, and impaired glucose tolerance. Thus, the 5-HT_{2C} receptor is reportedly involved in the serotonergic control of food intake and body weight. The knockout mice were also prone to spontaneous death from seizures, suggesting that 5-HT_{2C} receptors also mediate tonic inhibition of neuronal network excitability. (Tecott L H, et al. Eating disorder and epilepsy in mice lacking 5-HT_{2C} serotonin receptors. Nature. 1995, 374(6522):542-6).

Without being bound by theory, the adverse effects associated with the use of fenfluramine as an anorexic agent are thought to be attributable to the interaction of fenfluramine's major metabolite norfenfluramine with the 5-HT_{2B} receptor, which is associated with heart valve hypertrophy.

Fenfluramine and its major metabolite, norfenfluramine, were reported to be potent substrates for norepinephrine transporters. (Rothman, et al., J. Pharmacol. Exp. Ther. 305(3):1191-9). Fenfluramine also acts as a norepinephrine releasing agent to a lesser extent, particularly via its active metabolite norfenfluramine. Fenfluramine causes the release of serotonin by disrupting vesicular storage of the neurotransmitter and reversing serotonin transporter function. At high concentrations, norfenfluramine also acts as a dopamine releasing agent, and so fenfluramine may do this at very high doses as well. In addition to monoamine release, while fenfluramine binds only very weakly to the serotonin 5-HT₂ receptors, norfenfluramine binds to and activates the serotonin 5-HT_{2B} and 5-HT_{2C} receptors with high affinity and the serotonin 5-HT_{2A} receptor with moderate affinity. The result of the increased serotonergic and noradrenergic neurotransmission is a feeling of fullness and reduced appetite.

Despite past cardiovascular safety concerns that arose when high doses of fenfluramine were used for treatment of adult obesity, attempts have been made to identify further therapeutic uses for that product, while weighing the known cardiovascular risks of fenfluramine against potential therapeutic benefits. One disorder for which new treatment options are sorely needed is epilepsy or epileptic encephalopathy, and in particular, epilepsy syndromes which are refractory to known treatments. Epilepsy is a functional disturbance of the central nervous system (CNS) induced by abnormal electrical discharges and marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

Although some antiepileptic drugs have been developed, approximately one third of patients with epilepsy are refractory to treatment. Therefore, the search for new mechanisms and medications that can regulate cellular excitability continues. Three drugs that are especially effective for partial onset seizures are vigabatrin, a selective and irreversible GABA-transaminase inhibitor that greatly increases whole-brain levels of GABA; tiagabine, a potent inhibitor of GABA uptake into neurons and glial cells; and topiramate, which is believed to produce its antiepileptic effect through several mechanisms, including modification of Na⁺-dependent and/or Ca²⁺-dependent action potentials, enhancement of GABA-mediated Cl⁻ fluxes into neurons, and inhibition of kainate-mediated conductance at glutamate receptors of the AMPA/kainate type. (Angehagen, et al., 2003, Neurochemical Research, 28(2):333-340).

Historically, investigation of fenfluramine's efficacy in epilepsy patients led to a common paradigm, i.e., that fenfluramine's primary effects were on behaviors that caused or induced seizures, not treating or preventing the seizure itself.

For example, Aicardi and Gastaut (New England Journal of Medicine (1985), 313:1419 and Archives of Neurology (1988) 45:923-925) reported four cases of self-induced photosensitive seizures, i.e., seizures caused by patients purposely staring into bright lights or the sun, which were found to respond to treatment with fenfluramine.

Clemens, in Epilepsy Research (1988) 2:340-343, reported a case study wherein a boy suffering pattern sensitivity-induced seizures that were resistant to anticonvulsive treatment was treated with fenfluramine to curb the patient's compulsive seizure-inducing behavior. Fenfluramine reportedly successfully terminated these self-induced seizures. Clemens concluded that this was because fenfluramine blocked the photosensitive triggering mechanism, and, secondarily, by diminishing the pathological drive toward the seizure triggering behavior/compulsion, i.e., not by treating the seizure itself.

In Neuropaediatrics, (1996); 27(4):171-173, Boel and Casaer reported on a study on the effects of fenfluramine on children with refractory epilepsy, all of whom exhibited compulsive seizure-inducing behavior. They observed that when fenfluramine was administered at a dose of 0.5 to 1 mg/kg/day, this resulted in a reduction in the number of seizures experienced by the patients, and concluded that “this drug could have significant anti-epileptic activity in a selected group of young patients with idiopathy or symptomatic generalized epilepsy, namely, children with self-induced seizures.” The authors noted that “[i]t may well be that fenfluramine has no direct antiepileptic activity but acts through its effect on the compulsion to induce seizures.” Hence, the authors suggested that fenfluramine affected behavior and not the seizure itself.

In a letter to Epilepsia, published in that journal (Epilepsia, 43(2):205-206, 2002), Boel and Casaer commented that fenfluramine appeared to be of therapeutic benefit in patients with intractable epilepsy and self-induced seizures. However, the authors did not attribute fenfluramine's efficacy to generalized anti-seizure activity.

A large number of subtypes of epilepsy or epileptic encephalopathy have been characterized, each with its own unique clinical symptoms, signs, and phenotype, underlying pathophysiology and distinct responses to different treatments. The present disclosure has applicability with respect to a range of different types of epilepsies and epilepsy subtypes, including Dravet syndrome, Doose syndrome, infantile spasms, and Lennox-Gastaut syndrome. There are a large number of subtypes of epilepsy that have been characterized. For example, the most recent classification system, and one that is widely accepted in the art, is that adopted by the International League Against Epilepsy's (“ILAE”) Commission on Classification and Terminology [See e.g., Berg et al., “Revised terminology and concepts for organization of seizures,” Epilepsia, 51(4):676-685 (2010)]:

I. Electroclinical syndromes arranged by age at onset:
A. Neonatal period (1. Benign familial neonatal epilepsy (BFNE), 2. Early myoclonic encephalopathy (EME), 3. Ohtahara syndrome),
B. Infancy (1. Epilepsy of infancy with migrating focal seizures, 2. West syndrome, 3. Myoclonic epilepsy in infancy (MEI), 4. Benign infantile epilepsy, 5. Benign familial infantile epilepsy, 6. Dravet syndrome, 7. Myoclonic encephalopathy in nonprogressive disorders),
C. Childhood (1. Febrile seizures plus (FS+) (can start in infancy), 2. Panayiotopoulos syndrome, 3. Epilepsy with myoclonic atonic (previously astatic) seizures, 4. Benign epilepsy with centrotemporal spikes (BECTS), 5. Autosomal-dominant nocturnal frontal lobe epilepsy (ADNFLE), 6. Late onset childhood occipital epilepsy (Gastaut type), 7. Epilepsy with myoclonic absences, 8. Lennox-Gastaut syndrome, 9. Epileptic encephalopathy with continuous spike-and-wave during sleep (CSWS), 10. Landau-Kleffner syndrome (LKS), 11. Childhood absence epilepsy (CAE));
D. Adolescence—Adult (1. Juvenile absence epilepsy (JAE), 2. Juvenile myoclonic epilepsy (JME), 3 Epilepsy with generalized tonic-clonic seizures alone, 4. Progressive myoclonus epilepsies (PME), 5. Autosomal dominant epilepsy with auditory features (ADEAF), 6. Other familial temporal lobe epilepsies,
E. Less specific age relationship (1. Familial focal epilepsy with variable foci (childhood to adult), 2. Reflex epilepsies);

II. Distinctive constellations:
A. Mesial temporal lobe epilepsy with hippocampal sclerosis (MTLE with HS),
B. Rasmussen syndrome,
C. Gelastic seizures with hypothalamic hamartoma,
D. Hemiconvulsion-hemiplegia-epilepsy,
E. Other epilepsies, distinguished by 1. presumed cause (presence or absence of a known structural or metabolic condition, then 2. primary mode of seizure onset (generalized vs. focal);

III. Epilepsies attributed to and organized by structural-metabolic causes:
A. Malformations of cortical development (hemimegalencephaly, heterotopias, etc.),
B. Neurocutaneous syndromes (tuberous sclerosis complex, Sturge-Weber, etc.),
C. Tumor,
D. Infection,
E. Trauma;

IV. Angioma: A. Perinatal insults, B. Stroke, C. Other causes;

V. Epilepsies of unknown cause;

VI. Conditions with epileptic seizures that are traditionally not diagnosed as a form of epilepsy per se; A. Benign neonatal seizures (BNS); and B. Febrile seizures (FS).

See Berg et. al, “Revised terminology and concepts for organization of seizures,” Epilepsia, 51(4):676-685 (2010)).

Part V of the ILAE classification scheme underscores the fact that the list is far from complete, and that there are still subtypes of epilepsy that have not yet been fully characterized, or that remain unrecognized as distinct syndromes.

Those skilled in the art will recognize that different subtypes of epilepsy are triggered by different stimuli, are controlled by different biological pathways, and have different causes, whether genetic, environmental, and/or due to disease or injury of the brain. In other words, the skilled artisan will recognize that teachings relating to one epileptic subtype are most commonly not necessarily applicable to other subtypes. Of particular importance is the fact that there are a large number of compounds that are used to treat different types of epilepsy, and different epilepsy subtypes respond differently to different anticonvulsant drugs. That is, while a particular drug may be effective against one form of epilepsy, it may be wholly ineffective against others, or even contra-indicated due to exacerbation of symptoms, such as worsening the frequency and severity of the seizures. As a result, efficacy of a particular drug with respect to a particular type of epilepsy is wholly unpredictable, and the discovery that a particular drug is effective in treating in treating a type of epilepsy for which that drug was not previously known to be effective is nearly always surprising, even in cases where the drug is known to be effective against another epilepsy type. Furthermore, as will be described in detail below, effective treatment of a form of epilepsy with fenfluramine can contra-indicate co-administration of and/or treatment with other therapeutic agents.

Examples of such serotonergic drugs are the (selective) serotonin reuptake inhibitors ((S)SRIs), such as for example, fluoxetine, sertraline, paroxetine, the tetracyclic antidepressants (TeCAs, for example, mirtazapine), the tricyclic antidepressants (TCAs), such as, for example, imipramine or doxepin, or direct 5-HT receptor agonists, two examples of which are lorcaserin (selective for 5-HT_{2C}) or buspirone (a selective partial agonist at 5-HT_{1A}) do not fully explain fenfluramine's activity in refractory epilepsy.

The present disclosure provides the insight that certain centrally active (CNS penetrant) drugs administered during fenfluramine therapy decrease fenfluramine's protection against seizures.

Several complicating factors in predicting CNS penetrance is blood brain barrier (BBB) dysfunction caused by seizures, especially treatment resistant seizures and epileptic encephalopathies and drug efflux transporters in the BBB. Some polymorphisms in for example MDR1 and P-gp genes have been linked with lower expression or functional activity of these efflux transporters in the blood brain barrier resulting in higher levels of drugs reaching the brain. Conversely polymorphisms having increased affinities for certain drugs been implicated in refractory epilepsies [Siddiqui, et al., N Eng. J Med 2003; 348:1442-1448].

### Patients

A method of the present invention can be practiced on any appropriately diagnosed patient. In some embodiments, a patient to be treated in the context of the present disclosure is an adult patient (e.g., a human adult patient, e.g., 18 to 75 years old). In some embodiments, a patient to be treated in the context of the present disclosure is child (e.g., a human child, e.g., 2 to 18 years old).

In alternate exemplary embodiments of the present invention, the patient is aged about 18 or less, about 16 or less, about 14 or less, about 12 or less, about 10 or less, about 8 or less, about 6 or less or about 4 or less to about 0 months or more, about 1 month or more, about 2 months or more, about 4 months or more, about 6 months or more or about 1 year or more. Thus, in some embodiments, a diagnosed patient is about one month old to about 18 years old when treated.

In some embodiments, a patient is also characterized by extreme weight loss, wasting, cachexia and/or loss of appetite.

In some embodiments, a patient is also characterized by a co-morbid psychiatric condition and/or psychosis. In some embodiments, a co-morbid psychiatric condition or psychosis is or comprises tangential, incoherent speech and thought, hallucinations, delusions, and aggression, as well as exaggerated, bizarre, disorganized behaviors, poverty of speech or speech content, flattened affect, social withdrawal, anhedonia, apathy, impaired attention, and/or impaired self-monitoring.

### Dravet Syndrome

In some embodiments, an epilepsy that may be treated with fenfluramine is Dravet syndrome. Dravet syndrome is a rare and catastrophic form of intractable epilepsy that begins in infancy. Children with Dravet syndrome do not outgrow the condition, and it affects every aspect of their daily lives, according to Dravet Foundation.org. Children with the seizure disorder also face behavior and developmental delays; movement and balance issues; bone problems; delayed language and speech problems; growth and nutrition issues; trouble sleeping; chronic infections; and problems regulating body temperature. People with this disorder also have a higher risk of death during seizures.

Initially, in the first year of life a patient with Dravet syndrome experiences prolonged seizures. In their second year, additional types of seizure begin to occur and this typically coincides with a developmental decline, possibly due to repeated seizures causing brain damage such as cerebral hypoxia. This then leads to poor development of cognition, language and motor skills.

Children with Dravet syndrome are likely to experience multiple seizures per day. Epileptic seizures are far more likely to result in death in sufferers of Dravet syndrome; approximately 10 to 15% of patients diagnosed with Dravet syndrome die in childhood, in some cases between two and four years of age. The mean age at death of patients is reported to be 8.7±9.8 years (SD), with 73% of deaths occurring before the age of 10 years, and 93% before the age of 20. Additionally, patients are at risk of numerous associated conditions including orthopedic developmental issues, impaired growth and chronic infections.

Of particular concern, children with Dravet syndrome are susceptible to episodes of Status Epilepticus, a convulsive seizure lasting longer than 5 minutes. This severe and intractable condition is categorized as a medical emergency requiring immediate medical intervention, typically involving hospitalization for intravenous anticonvulsant medication and/or medically-induced coma. Status epilepticus can be fatal. It can also be associated with severe cerebral hypoxia, possibly leading to damage to brain tissue. Frequent hospitalizations of children with Dravet syndrome are clearly distressing, not only to the patient but also to family and caregivers.

The cost of care for Dravet syndrome patients is also high as the affected children require constant supervision and many require institutionalization as they reach teenage years.

Seizures in Dravet syndrome can be difficult to manage but may be reduced by anticonvulsant medications such as clobazam, stiripentol, topiramate and valproate. Because the course of the disorder varies from individual to individual, treatment protocols may vary. A diet high in fats and low in carbohydrates may also be beneficial, known as a ketogenic diet. Although diet adjustment can help, it does not eliminate the symptoms. Until a better form of treatment or cure is discovered, those with this disease will have myoclonic epilepsy for the rest of their lives.

At present, although a number of anticonvulsant therapies can be employed to reduce the instance of seizures in patients with Dravet syndrome, the results obtained with such therapies are typically poor and those therapies only effect partial cessation of seizures at best. Seizures associated with Dravet syndrome are typically resistant to conventional treatments. Further, many anticonvulsants such as clobazam and clonazepam have undesirable side effects, which are particularly acute and prominent in pediatric patients.

Non-epileptic brains have a natural balance of excitation (that can evoke seizures) and inhibition (that can reduce seizures). Sodium channel blockers preferentially affect sodium channels at a specific stage of their cycle of rest, activation and inactivation, often by delaying the recovery from the inactivated state, thereby producing a cumulative reduction of Na+. Sodium channel blockers are widely used in treating epilepsies or epileptic encephalopathies that are caused by too much excitatory neurotransmission (with the exception of SCN1A-mutation-related epilepsies). In some epilepsies or epileptic encephalopathies, sodium channel blockers may work to correct an imbalance of excitatory and/or inhibitory neurotransmitter(s) to make seizures less likely to occur. However, while sodium channel blockers are beneficial in treatment of some epilepsies, this class of drugs are contra-indicated in Dravet syndrome, as sodium channel blockers have been found to lead to a greater incidence of seizures in almost all Dravet syndrome patients.

Without being bound by theory, approximately 70-90% of patients with Dravet syndrome have nonsense mutations in the SCN1A gene encoding neuronal voltage-gated sodium channel Na(V)1.1 and resulting in a loss of function of the sodium channel. Given that sodium channel blockers are reported to prevent seizure activity in some epilepsies, treating Dravet patients lacking SCN1A function with sodium channel blockers might be expected to prevent seizures in patients with Dravet syndrome. Instead, treatment of patients with Dravet syndrome with sodium channel blockers leads to increased seizure activity. One explanation may be that, in Dravet syndrome patients, the problem is not too much excitation, but rather too little inhibition. Therefore, giving sodium channel blocking drugs to Dravet syndrome patients decreases the amount of inhibitory neurotransmitters in the brain, tipping the balance toward more seizure activity. Thus, certain anticonvulsant drugs classed as Sodium Channel Blockers are now known to make seizures worse in most Dravet patients. Thus, according to the present disclosure, sodium channel blocker drugs may be contraindicated in connection with the present invention may include the following: phenytoin, carbamazepine, gabapentin, lamotrigine, oxcarbazepine, rufinamide, lacosamide, eslicarbazepine acetate, and fosphenytoin.

A subject with epilepsy or epileptic encephalopathy may have a mutation in one or more of a gene selected from the group consisting of SCN1A, SCN1B, SCN2A, SCN3A, SCN9A, GABRG2, GABRD and PCDH19.

In some embodiments, provided are methods for treating Dravet syndrome comprising administering a therapeutically effective amount of fenfluramine to a patient in need thereof, wherein the patient is not concurrently being administered and/or exposed to a serotonin (5-HT)-receptor antagonist. In some embodiments, provided are methods for treating weight loss, wasting, cachexia and/or loss of appetite in a population of Dravet syndrome patients being administered and/or exposed to fenfluramine or a pharmaceutically acceptable salt thereof, the method comprising: administering a serotonin (5-HT)-receptor antagonist to the patients, and providing to the patient instructions that the anti-seizure efficacy of fenfluramine or a pharmaceutically acceptable salt thereof may be reduced by administration of said serotonin (5-HT)-receptor antagonist. In certain embodiments, a Dravet syndrome patient is administered a 5-HT_{1A} serotonin receptor antagonist or a 5-HT_{2C} serotonin receptor antagonist to the patients. In certain embodiments, a Dravet syndrome patient is informed that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

In some embodiments, provided are kits for treating Dravet syndrome in accordance with provided methods.

### Lennox-Gastaut Syndrome

Another exemplary form of epilepsy that may be treated with fenfluramine is Lennox-Gastaut syndrome (LGS). LGS was first described in 1960, and named for neurologists William G. Lennox (Boston, USA) and Henri Gastaut (Marseille, France). It is a difficult-to-treat form of childhood-onset epilepsy that most often appears between the second and sixth year of life, although it can occur at an earlier or later age. LGS is characterized by frequent seizures and different seizure types; it is typically accompanied by developmental delay and psychological and behavioral problems. In children, common causes of LGS include perinatal brain injury, brain malformations such as tuberous sclerosis or cortical dysplasia, CNS infection, and degenerative or metabolic disorders of the nervous system.

Daily multiple seizures of different types are typical in LGS. Also typical is the broad range of seizures that can occur. The most common seizure types are tonic-axial, atonic, and absence seizures, but myoclonic, generalized tonic-clonic, and focal seizures can also occur in any LGS patient. Atonic, atypical absence, tonic, focal, and tonic-clonic seizures are also common. Additionally, many LGS patients will have status epilepticus, often of the nonconvulsive type, which is characterized by dizziness, apathy, and unresponsiveness. Further, most patients have atonic seizures, also called drop seizures, which cause their muscles to go limp and result in the patient suddenly and unexpectedly to fall to the ground, often causing significant injury, which is why patients often wear a helmet to prevent head injury.

In addition to daily multiple seizures of various types, children with LGS frequently have arrested/slowed psycho-motor development and behavior disorders.

The syndrome is also characterized by a specific finding on electroencephalogram (EEG), specifically an interictal (i.e., between-seizures) slow spike-wave complexes and fast activity during sleep.

### Diagnosis of LGS

LGS is a syndrome and hence its diagnosis is based on the presence of specific clinical symptoms, signs, and laboratory tests. LGS is typically identified by a triad of features including multiple types of seizures, mental retardation or regression and abnormal EEG with generalized slow spike and wave discharges. Physicians use EEG to assist in diagnosing LGS. Diagnosis may be difficult at the onset of the initial symptom(s) because the triad of features associated with LGS, such as tonic seizures, may not be fully established, and EEG during sleep is required to confirm the condition. Thus, even though there may be overlap in clinical presentation with other epilepsies, LGS is agreed to be a well-defined distinct diagnosis by both the International League Against Epilepsy (ILAE), considered the world's leading expert medical society on epilepsy, and the FDA.

The diagnosis of LGS is more obvious when the patient suffers frequent and manifold seizures, with the classic pattern on the electro-encephalogram (EEG), i.e., a slowed rhythm with Spike-wave-pattern, or with a multifocal and generalizing sharp-slow-wave-discharges at 1.5-2.5 Hz. During sleep, tonic patterns (fast activity) can often be seen.

General medical investigation usually reveals developmental delay and cognitive deficiencies in children with LGS. These may precede development of seizures, or require up to two years after the seizures begin, in order to become apparent.

There may be multiple etiologies for LGS, including genetic, structural, metabolic or unknown. Approximately one-quarter have no prior history of epilepsy, neurological abnormality or developmental delay prior to the onset of LGS symptoms. Underlying pathologies causing LGS may include encephalitis and/or meningitis, brain malformations (e.g., cortical dysplasias), birth injury, hypoxia-ischemia injury, frontal lobe lesions, and trauma.

An important differential diagnosis is ‘Pseudo-Lennox-Syndrome’, also called atypical benign partial epilepsy of childhood, which differs from LGS, in that there are no tonic seizures; sleeping EEG provides the best basis for distinguishing between the two. In addition, ‘Pseudo-Lennox-Syndrome’ has an entirely different etiology and prognosis than LGS.

### Treatment of LGS

The optimum treatment for Lennox-Gastaut syndrome has yet to be established. Many different treatments are currently used in the treatment of this disorder and many more have been tried in the past, most often with little success.

A variety of therapeutic approaches are currently used in LGS, including conventional antiepileptic medications, diet and surgery, however the evidence supporting these therapies is not robust and treatment remains most often ineffective. The use of several common first-line treatments is based on clinical experience or conventional wisdom; examples include broad spectrum anti-convulsant medications, such as valproic acid, and benzodiazepines, most often clonazepam and clobazam. A few drugs have been proven effective for some patients for certain seizure types by double-blind placebo-controlled studies; examples include clobazam, lamotrigine, topiramate, felbamate, and rufinamide, although most patients continue to have significant seizures even while taking these medications. Second-line medications currently in use, such as zonisamide, are prescribed based on results of some open-label uncontrolled studies. The ketogenic diet may be useful in some patients with LGS refractory to medical treatment. Surgical options for LGS include corpus callostomy (for drop attacks), vagus nerve stimulation, and focal cortical resection (in the presence of a single resectable lesion). However, it should be noted that significant improvement from any of these therapies alone or in combination is a rare occurrence.

Despite the severity of LGS's symptoms and the frequency with which it occurs (it accounts for up to 10% of all childhood epilepsies), there is currently no standard evidence-based treatment for the disease. A comprehensive review of the literature [see Hancock E C & Cross J H, Treatment of Lennox-Gastaut syndrome (Review), published in The Cochrane Library 2013, Issue 2] discovered only nine randomized controlled trials which evaluated the pharmaceutical treatment of the syndrome. The authors concluded that there is a paucity of research and “ . . . that no monotherapy (to date) has been shown to be highly effective in this syndrome.” Id at page 12. The authors further concluded that “[t]he optimum treatment for LGS remains uncertain and no study to date has shown any one drug to be highly efficacious”. Id at page 12.

Without being bound by theory, fenfluramine has been known to trigger the release of serotonin (5-HT) in the brain due to disruption of its vesicular storage and to inhibit serotonin reuptake. Fenfluramine's mechanism of action made it suitable for the treatment of epilepsy. In fact, there are no scientific publications demonstrating or even hypothesizing that 5-HT abnormalities are a possible underlying pathophysiologic cause for LGS or are causally related to the associated seizures in this specific epilepsy condition. Furthermore, since there has been no scientific hypothesis relating serotonin abnormalities in LGS, there are no studies nor even individual case reports in the medical literature which describe attempts to treat LGS using medications that interacts with serotonin. The lack of data or even speculation in the literature regarding the use of fenfluramine or serotonergic agents in general to treat LGS are facts that strongly support the unexpected nature of this invention: given that LGS is a devastating refractory epilepsy condition and the number of people affected, investigators would be strongly motivated to investigate any treatment they perceived as having any potential for efficacy.

Thus, one method of treating epilepsy and/or epileptic encephalopathy may be to stimulate one or more 5-HT receptors in the brain of a patient may be to administer an effective dose of fenfluramine to said patient, said one or more 5-HT receptors being selected from one or more of 5-HT₁, 5-HT_{1A}, 5-HT_{1B}, 5-HT_{1C}, 5-HT_{1D}, 5-HT_{1E}, 5-HT_{1F}, 5-HT₂, 5-HT_{2A}, 5-HT_{2B}, 5-HT_{2C}, 5-HT₃, 5-HT₄, 5-HT₅, 5-HT_{5A}, 5-HT_{5B} 5-HT₆, and 5-HT₇ amongst others. In certain embodiments of this disclosure, the patient has been diagnosed with epilepsy.

Thus, fenfluramine has therapeutic use in the treatment of certain epilepsies and/or epileptic encephalopathies. However, the anorectic effects of fenfluramine may exacerbate symptoms of some forms of epilepsy in which extreme weight loss, wasting and/or cachexia are a problem. For example, in some forms of epilepsy, patients who are being treated with fenfluramine to ameliorate symptoms of epilepsy (such as seizures) may exhibit severe weight loss, anorexia and/or wasting as characteristics of the epilepsy, or as side effects of the fenfluramine treatment. Due to its appetite stimulating effects, histamine antagonists might be considered by a treating physician to be useful to counteract severe weight loss, wasting and/or anorexia in these epileptic patients with being treated with fenfluramine. Centrally acting H1 antagonists are also associated with sedation an drowsiness and some are administered to help with sleep induction of maintenance, for example doxepin (Silenor®) is a potent H1 antagonist used for help with sleep maintenance.

In some embodiments, provided are methods for treating LGS comprising administering a therapeutically effective amount of fenfluramine to a patient in need thereof, wherein the patient is not concurrently being administered and/or exposed to a serotonin (5-HT)-receptor antagonist. In some embodiments, provided are methods for treating weight loss, wasting, cachexia and/or loss of appetite in a population of LGS patients being administered and/or exposed to fenfluramine or a pharmaceutically acceptable salt thereof, the method comprising: administering a serotonin (5-HT)-receptor antagonist to the patients, and providing to the patient instructions that the anti-seizure efficacy of fenfluramine or a pharmaceutically acceptable salt thereof may be reduced by administration of said serotonin (5-HT)-receptor antagonist. In some certain embodiments, a LGS patient is administered a 5-HT_{1A} serotonin receptor antagonist or a 5-HT_{2C} serotonin receptor antagonist to the patients. In some certain embodiments, a LGS patient is informed that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

In some embodiments, provided are kits for treating LGS in accordance with provided methods.

### Rett Syndrome

In some embodiments, epilepsy and/or epileptic encephalopathy in the context of the present disclosure is or comprises Rett syndrome. Rett syndrome is a rare neurodevelopmental disorder caused by a mutation in the methyl CpG binding protein 2 (MECP2) gene in which the phenotype is characterized by a period of normal development in the infant, soon followed by regression of growth and cognitive and motor development and onset of seizures. During the period of regression patients start to exhibit autistic features, loss of acquired speech and social interaction, and purposeful use of the hands, such use being replaced with repetitive stereotypic movements. Eventually patients exhibit moderate to profound mental retardation.

In some embodiments, provided are methods for treating Rett syndrome comprising administering a therapeutically effective amount of fenfluramine to a patient in need thereof, wherein the patient is not concurrently being administered and/or exposed to a serotonin (5-HT)-receptor antagonist. In some embodiments, provided are methods for treating weight loss, wasting, cachexia and/or loss of appetite in a population of Rett syndrome patients being administered and/or exposed to fenfluramine or a pharmaceutically acceptable salt thereof, the method comprising: administering a serotonin (5-HT)-receptor antagonist to the patients, and providing to the patient instructions that the anti-seizure efficacy of fenfluramine or a pharmaceutically acceptable salt thereof may be reduced by administration of said serotonin (5-HT)-receptor antagonist. In some certain embodiments, a Rett syndrome patient is administered a 5-HT_{1A} serotonin receptor antagonist or a 5-HT_{2C} serotonin receptor antagonist to the patients. In some certain embodiments, a Rett syndrome patient is informed that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

In some embodiments, provided are kits for treating Rett syndrome in accordance with provided methods.

### Sertononeric Antagonists

Methods and kits of the present disclosure encompass a recognition that at least certain serotonergic antagonists are contraindicated with administration of fenfluramine. Accordingly, the present disclosure provides methods where patients being administered and/or exposed to fenfluramine have been warned against co-administration of certain serotonin receptor antagonists, are not co-administered a serotonin receptor antagonist, and/or in which co-administration of a serotonin-receptor antagonist is discontinued.

Cyproheptadine (C₂₁H₂₁N) is a histamine and serotonergic 5-HT_{2A} and 5-HT_{2C} antagonist in the family of drugs called antihistamines, and is used orally (often as a salt of the synthetic methyl-piperidine derivative, C₂₁H₂₁N.HCl) to treat asthma, allergy and cold symptoms (such as sneezing and runny nose), and to relieve itching caused by hives and other skin disorders. Cyproheptadine is also used in patients with anorexia to stimulate appetite.

Cyproheptadine competes with histamine for binding at histamine-receptor sites, thereby competitively antagonizing histamine stimulation of histamine-receptors in the gastrointestinal tract, large blood vessels, and bronchial smooth muscle. In addition to its antihistamine action, cyproheptadine also competes with serotonin for binding at serotonin receptor sites, and has anticholinergic, antiserotonergic, antidopaminergic, and local anesthetic and sedative properties. One of the side effects of cyproheptadine is increased appetite and weight gain, which has led to its off-label use to stimulate appetite and weight gain in children who are wasting, in patients with anorexia, in people with cystic fibrosis and in patients exhibiting weight loss and/or cachexia caused by cancer and its treatment.

For example, the effect of cyproheptadine on water and food intake and on body weight has been studied in fasted adult and weanling rats; a reduction of serotonin system activity was observed to increase food intake. (Ghosh M N, Parvathy S. Br. J. Pharmacol. 1973, 48(2):328P-329P).

Other appetite stimulants have been used as part of an array of treatment for anorexia and weight loss. Multiple agents primarily prescribed for a different treatment also may have a secondary effect on appetite stimulation; such agents include hormones (ghrelin, growth hormone, insulin), antihistamines (cyproheptadine and pizotifen), steroids (megesterol acetate (MA), oxandrolone, prednisone), cannabinoids (dronabinol), antidepressants (mirtazapine) and antipsychotics (quetiapine, olanzapine, risperdone). (Chinuck, et al., 2014, Cochrane Database of Systematic Reviews, Issue 7. Art. No.: CD008190).

For example, dronabinol is the principal psychoactive substance present in marijuana. Capsules of synthetic tetrahydrocannabinol (THC) (dronabinol) have been available for restricted medical use in the USA since 1985. Nabilone, a synthetic THC analogue taken orally, is the only cannabinoid licensed for prescription in the UK for the treatment of nausea and vomiting caused by chemotherapy; its use in other indications is only possible on a ‘named patient’ basis if the drug is supplied by a hospital pharmacy (EMC 2014a). Dronabinol has been shown to be effective as an oral appetite stimulant in HIV and cancer patients using doses of 2.5 mg to a maximum of 5 mg twice daily (Anstead, et al., “Dronabinol, an effective and safe appetite stimulant in cystic fibrosis [abstract].” 2003, Pediatric Pulmonology 36(25):343).

It has been discovered and described herein that serotonin receptor antagonists, particularly those blocking 5-HT_{1A}, 5-HT_{1D}, 5-HT_{2B}, and 5-HT_{2C} receptor subtypes inhibit the seizure-blocking activity of fenfluramine. Therefore, as set forth herein, in epileptic patients being treated with fenfluramine, co-administration of potent serotonin receptor antagonists (whether concurrently or subsequently, in either order of administration) is should be avoided or used with caution and/or close monitoring in the course of treatment, amelioration and/or prevention of symptoms of epilepsy or epileptic encephalopathy.

Suitable alternatives include CB1 agonists that may be useful for treating weight loss, wasting and/or loss of appetite in these patients include (but are not limited to):5F-AB-PINACA, AB-PINACA, AM-1220, AM-1221, AM-2201, Anandamide, N-Arachidonoyl, dopamine, 2-Arachidonoylglycerol, 2-Arachidonyl glyceryl ether, Cannabinol, CP 47,497, CP 55,940, Epicatechin gallate, Gallocatechol, JWH-015, JWH-018, JWH-073, JWH-081, JWH-122, Kava, L-759,633, Levonantradol, Tetrahydrocannabinol, WIN 55,212-2, and Yangonin.

The present disclosure is generally directed to kits and methods of treating and/or preventing one or more symptoms of epilepsy or epileptic encephalopathy in a patient comprising administering an effective dose of fenfluramine alone or in combination with one or more drugs as described herein, wherein the patient is not also being treated with a serotonergic antagonist.

Potency of a drug, in terms of receptor binding, and particularly antagonist binding, is a function of an antagonist's affinity for the receptor in competition with a known agonist molecule. Competitive antagonists have no ability to activate the receptors (efficacy) they bind, but rather exert their effect by blocking the action of an agonist. The potency of an antagonist is usually defined by its half maximal inhibitory concentration (IC50). Affinity of an antagonist for a receptor can be determined by converting an IC50 value to an inhibition constant, Ki, using the Cheng-Prusoff equation. Since the Ki takes into account the IC50 in its calculation, the Ki is commonly reported in the scientific literature. Lower IC50 and Ki values indicate a drug having more potency and affinity for a particular receptor. For a competitive inhibitor the IC50 is always greater than Ki. If the Ki or IC50 values at a receptor are approximately the same or less than the drug levels achieved in a patient there is a likelihood of a pharmacological effect. In some instances, the pharmacological effect is noticeable. For instance, drugs with antagonist potency at muscarinic receptors can cause common side effects such as dry mouth, hyperthermia, flushing and blurred vision; these are common side effects that a physician recognizes and associates with anti-muscarinic activity. In some instances, a potent antagonist at one or more receptors does not usually cause noticeable side effects in the patient population being treated. In this second instance, a treating physician may not recognize or be able to associate a potent antagonist with a potential off-target effect through either clinical experience or training.

In some embodiments, a 5-HT receptor antagonist is selected from Table 3.

TABLE 3
Potent serotonergic (5-HT) antagonists with a Ki of ≤3 nM

| 5-HT_{2A} | 5-HT_{2C} | 5-HT_{1A} | 5-HT_{1D} |
|---|---|---|---|
| ALTANSERIN | AMOXAPINE | ARIPIPRAZOLE | ERGOTAMINE |
| AMITRIPTYLINE | CLOZAPINE | CYANOPINDOLOL | LISURIDE |
| AMOXAPINE | CYPROHEPTADINE | ERGOTAMINE | LYSERGOL |
| BENPERIDOL | ISOCLOZAPINE | FLESINOXAN | METERGOLINE |
| BUTACLAMOL, (+) | ISOCLOZAPINE | IPSAPIRONE | METHIOTHEPIN |
| BUTACLAMOL. d- | MESULERGINE | Lecozotan | Naratriptan |
| CHLORPROMAZINE | METERGOLINE | LISURIDE | OXYMETAZOLINE |
| CHLORPROTHIXENE | METERGOLINE | Roxindole | SUMATRIPTAN |
| CINANSERIN | METHIOTHEPIN | SPIROXATRINE | Ziprasidone |
| CLOPIPAZAN | METHIOTHEPIN | Urapidil-5-methyl | |
| CLOZAPINE | METHYSERGIDE | Vilazodone | |
| Cyamemazine | METITEPIN | Ziprasidone | |
| CYPROHEPTADINE | MIANSERIN | | |
| DROPERIDOL | OLANZAPINE | | |
| ERGOTAMINE | ORG-5222 - | | |
| FLUPENTHIXOL, | Asenapine | | |
| Alpha | SERTINDOLE | | |
| FLUPHENAZINE | Ziprasidone | | |
| FLUSPIPERONE | ZOTEPINE | | |
| ILOPERIDONE | | | |
| ISOCLOZAPINE | | | |
| KETANSERIN | | | |
| LISURIDE | | | |
| LOXAPINE | | | |
| Lurasidone | | | |
| MESULERGINE | | | |
| METERGOLINE | | | |
| METHERGINE | | | |
| METHIOTHEPIN | | | |
| METHYLERGONOVINE | | | |
| METHYSERGIDE | | | |
| METITEPIN | | | |
| MIANSERIN | | | |
| mirtazapine | | | |
| OCTOCLOTHEPIN | | | |
| OLANZAPINE | | | |
| ORG-5222 Asenapine | | | |
| Perospirone | | | |
| PIPAMPERONE | | | |
| PIRENPERONE | | | |
| PRAZOSIN | | | |
| PROPERICIAZINE | | | |
| RILAPINE | | | |
| RISPERIDONE | | | |
| RITANSERIN | | | |
| RMI 81,582 | | | |
| SERTINDOLE | | | |
| SETOPERONE | | | |
| SPIPERONE | | | |
| SPIPERONE, N-Me | | | |
| THIORIDAZINE | | | |
| THIOTHIXENE.cis- | | | |
| TIOSPERONE | | | |
| XYLAMIDINE | | | |
| Ziprasidone | | | |
| ZOTEPINE | | | |

In some embodiments, a serotonin receptor antagonist is a 5-HT_{1D} serotonin receptor antagonist selected from: ergotamine, lisuride, lysergol, metergoline, methiothepin, naratriptan, oxymetazoline, sumatriptan, and ziprasidone.

In some embodiments, a serotonin receptor antagonist is a 5-HT_{2A} serotonin receptor antagonist selected from: altanserin, amitriptyline, amoxapine, benperidol, (+)-butaclamol, d-butaclamol, chlorpromazine, chlorprothixene, cinanserin, clopipazan, clozapine, cyamemazine, cyproheptadine, droperidol, ergotamine, alpha-flupenthixol, fluphenazine, fluspiperone, iloperidone, isoclozapine, ketanserin, lisuride, loxapine, lurasidone, mesulergine, metergoline, methergine, methiothepin, methylergonovine, methysergide, metitepin, mianserin, mirtazapine, octoclothepin, olanzapine, ORG-5222 asenapine, perospirone, pipamperone, pirenperone, prazosin, propericiazine, rilapine, risperidone, ritanserin, RMI 81,582, sertindole, setoperone, spiperone, N-Me-spiperone, thioridazine, cis-thiothixene, tiosperone, xylamidine, ziprasidone, and zotepine.

### Kits

One aspect of the invention is a kit comprising a fenfluramine formulation, a package, and a package insert comprising content warning against co-administration with a serotonin (5-HT)-receptor antagonist.

In some embodiments of the kit, the 5-HT receptor antagonist is a 5-HT_{2A} receptor antagonist.

In some embodiments of the kit, the 5-HT receptor antagonist is a 5-HT_{2C} receptor antagonist.

Another aspect of the invention is a kit including a container comprising a plurality of doses of a formulation including a pharmaceutically acceptable carrier and an active ingredient comprising fenfluramine; and instructions for treating the patient diagnosed with epilepsy or epileptic encephalopathy, and the instructions include administering the formulation to the patient if the patient is not also being treated with a serotonin receptor antagonist.

In some embodiments of the kit, the formulation is an oral solution comprising 2.5 milligram of fenfluramine in each milliliter of liquid solution; and the instructions indicate dosing the patient based on patient weight and volume of oral solution administered.

In some embodiments of the kit, the formulation is a solid oral formulation selected from the group consisting of: a tablet, a disintegrating table, a capsule, a lozenge, and a sachet.

In some embodiments of the kit, the formulation is provided in a transdermal patch.

### Methods

In some embodiments, the present disclosure provides a method of treating, ameliorating and/or preventing a symptom of epilepsy or epileptic encephalopathy, comprising administering a therapeutically effective dose of fenfluramine or a pharmaceutically acceptable salt thereof to a patient or population of patients diagnosed with epilepsy or epileptic encephalopathy. In some embodiments, said patient(s) are informed that certain 5-HT serotonin receptor antagonists may impair fenfluramine's anti-epileptic activity. In some embodiments, said patient(s) are not co-administered certain 5-HT serotonin receptor antagonists. In some embodiments, 5-HT serotonin receptor antagonists are or comprise cyproheptadine, or a 5-HT_{1A} serotonin receptor antagonist, a 5-HT_{1D} serotonin receptor antagonist, a 5-HT_{2A} serotonin receptor antagonist, or a 5-HT_{2C} serotonin receptor antagonist. In some embodiments, a 5-HT receptor antagonist is selected from Table 3.

In some embodiments, the dose is in a range of from 10.0 mg/kg/day to 0.1 mg/kg/day.

In some embodiments, the dose is administered in a dosage form selected from the group consisting of oral, injectable, transdermal, inhaled, nasal, rectal, vaginal and parenteral delivery.

In some embodiments, the dosage form is an oral solution in an amount selected from the group consisting of 120 mg or less, 60 mg or less, 30 mg or less, and 20 mg or less.

In some embodiments, the co-therapeutic agent is a combination of stiripentol, clobazam, and valproate.

In some embodiments, the administering is over a period of months.

In some embodiments, the symptom is seizure, and the fenfluramine is formulated with a pharmaceutically acceptable carrier and an effective dose is less than 10.0 mg/kg/day to 0.01 mg/kg/day.

In some embodiments, the daily dose is selected from the group consisting of 60 mg or less, 30 mg or less, and 20 mg or less, and wherein the dose is administered in a dosage form selected from the group consisting of forms for oral, injectable, transdermal, inhaled, nasal, buccal, rectal, vaginal and parenteral delivery.

In some embodiments, the patient has been diagnosed with Dravet syndrome or Lennox-Gastaut syndrome. In some embodiments, the patient has been diagnosed with Lennox-Gastaut syndrome.

In some embodiments, the method further includes repeating the administering over a period of days until the patient exhibits a ≥40% reduction from baseline in convulsive seizure frequency.

In some embodiments, the method further includes repeating the administering until the patient is seizure free for a period of ≥1 day.

In some embodiments, the method further includes repeating the administering until the patient is seizure free for a period of ≥1 week.

In some embodiments, the method further includes repeating the administering until the patient is seizure free for a period of ≥1 month.

In some embodiments, the method further includes repeating the administering until the patient is seizure free for a period of ≥1 year.

In some embodiments, the method further includes repeating the administering until the patient is permanently seizure free.

In some embodiments of the method, the patient is age is between 3 and 18 years.

In another aspect, the invention provides a method of treating a symptom of epilepsy or epileptic encephalopathy in a patient diagnosed with epilepsy or epileptic encephalopathy, comprising administering a therapeutically effective dose of fenfluramine or a pharmaceutically acceptable salt thereof to the patient, wherein the patient is advised by a label, a package insert or a medication guide accompanying the fenfluramine to avoid treatment with a serotonin receptor antagonist.

In another aspect, the invention provides a method of treating a patient diagnosed with (a) Dravet syndrome or Lennox-Gastaut syndrome, and (b) weight loss, wasting and/or loss of appetite, by administering to the patient a liquid formulation comprising a pharmaceutically acceptable carrier and a therapeutically effective amount of fenfluramine or a pharmaceutically acceptable salt thereof; and advising the patient by a label, a package insert or a medication guide accompanying the formulation to avoid treatment with cyproheptadine.

In some embodiments, the patient has not been treated with cyproheptadine within 24 hours of treatment with fenfluramine.

In another aspect, the invention provides a kit, comprising a container comprising a plurality of doses of a formulation comprising a pharmaceutically acceptable carrier and an active ingredient comprising fenfluramine; and instructions for treating a patient diagnosed with epilepsy or epileptic encephalopathy, wherein the instructions include administering the formulation to the patient, and advising the patient to avoid treatment with serotonin receptor antagonists.

In another aspect, the invention provides a method, comprising administering to a patient diagnosed with Dravet syndrome or Lennox-Gastaut syndrome a liquid formulation comprising a pharmaceutically acceptable carrier and a therapeutically effective amount of fenfluramine or a pharmaceutically acceptable salt thereof, and advising the patient by a label accompanying the formulation to avoid treatment with serotonin receptor antagonists.

Some therapeutic agents may be effective in stimulating one or more 5-HT receptors in the brain of a patient by administering an effective dose of fenfluramine or a pharmaceutically acceptable salt thereof to that patient. Illustrative one or more 5-HT receptors are selected from the group consisting of one or more of 5-HT₁, 5-HT_{1A}, 5-HT_{1B}, 5-HT_{1C}, 5-HT_{1D}, 5-HT_{1E}, 5-HT_{1F}, 5-HT₂, 5-HT_{2A}, 5-HT_{2B}, 5-HT_{2C}, 5-HT₃, 5-HT₄, 5-HT₅, 5-HT_{5A}, 5-HT_{5B} 5-HT₆, and 5-HT₇. In addition, there may be non-5-HT binding in the brain including Sigma-1, M1 muscarinic, B-adrenergic.

In some embodiments, a 5-HT receptor antagonist is selected from Table 3.

Exemplary co-therapeutic agents for co-administration with the fenfluramine can be selected from the group consisting of cannabidiol, carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam. Use of a pharmaceutically acceptable salt of a co-therapeutic agent is also contemplated.

Similarly, selective GABA reuptake inhibitors/GABA-transaminase inhibitors including tiagabine and vigabatrin should be avoided in Dravet syndrome.

A double-blind placebo trial was performed using stiripentol, a GABAergic agent and as a positive allosteric modulator of GABAA receptor. This drug showed efficacy in trials, found to improve focal refractory epilepsy, as well as Dravet syndrome, supplemented with clobazam and valproate.

Stiripentol was found to reduce overall seizure rate by 70%, and is approved in Europe, Canada, Japan and Australia but not in the US, for the treatment of Dravet syndrome. Although stiripentol has some anticonvulsant activity on its own, it acts primarily by inhibiting the metabolism of other anticonvulsants thereby prolonging their activity. It is labeled for use in conjunction with clobazam and valproate. However, concerns remain regarding the use of stiripentol due to its inhibitory effect on hepatic cytochrome P450 enzymes. Further, the interactions of stiripentol with a large number of drugs means that combination therapy (which is typically required for patients with Dravet syndrome) is problematic. Additionally, the effectiveness of stiripentol is limited, with few if any patients ever becoming seizure free.

In some embodiments, fenfluramine may be co-administered with other known pharmaceutical drugs such as a co-therapeutic agent selected from the group consisting of carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam, or a pharmaceutically acceptable salt thereof.

The co-therapeutic agents have recommended dosing amounts. Those recommended dosing amounts are provided within the most current version of the Physician's Desk Reference (PDR) or online at emedicine.medscape.com, both of which are incorporated herein by reference specifically with respect to the co-therapeutic agents listed above and more specifically with respect to the dosing amounts recommended for those drugs.

In connection with the present invention, the co-therapeutic agent can be used in the recommended dosing amount or can be used in a range of from 100^{th} to 100 times 1/10 to 10 times ⅕ to 5 times ½ to twice the recommended dosing amount or any incremental 1/10 amount in between those ranges.

As a specific example of a combination of co-therapeutic agents with fenfluramine, the co-therapeutic agent may be any one of or all three of stiripentol, clobazam, and valproate. The fenfluramine may be administered in the amount of 0.8 mg/kg of patient body weight and co-administered with 3500 mg of stiripentol, 20 mg of clobazam, and 25 mg per kg of valproate. Each of those amounts may be increased to twice, three times, five times, or ten times that amount or decreased by 10%, 50%, or 75%.

Described herein is a method of treating the symptoms of epilepsy or epileptic encephalopathy in a patient diagnosed with epilepsy or epileptic encephalopathy, comprising administering an effective dose of fenfluramine or pharmaceutically acceptable salt to the patient, wherein the dose is administered in an amount in the range of from 10.0 mg/kg/day to about 0.01 mg/kg/day, or administered at 120 mg or less; or 60 mg or less; or 30 mg or less; or 20 mg or less, and may be administered in the absence of the administration of any other pharmaceutically active compound. In some embodiments, a therapeutically effective total daily dose of fenfluramine is no more than 40 mg, no more than 30 mg, or no more than 20 mg.

In some embodiments, the method is carried out wherein the effective dose is administered in a form selected from the group consisting of oral, injectable, transdermal, buccal, inhaled, nasal, rectal, vaginal, or parental, and wherein the formulation is oral, the formulation may be liquid which may be a solution or a suspension may be present within a container closed with a cap connected to a syringe graduated to determine the volume extracted from the container wherein the volume extracted relates to the amount of fenfluramine in a given liquid volume of formulation e.g. one milliliter of formulation contains 2.5 mg of fenfluramine. In some embodiments of the kit or method, fenfluramine is administered in a solid oral formulation in the form of a tablet, capsule, lozenge, or sachet.

The method may be carried out as a co-treatment with a different pharmaceutically active compound. The method may be carried out in a process wherein the patient is first then subjected to a series of tests to confirm diagnoses of epilepsy or epileptic encephalopathy.

Various compounds have been tested for treating different types of epilepsy or epileptic encephalopathy, and different epilepsy subtypes respond differently to different anticonvulsant drugs. For example, cannabidiol (CBD) has received orphan drug status in the United States, for treatment of Dravet syndrome; cannabidiol has been studied for treatment of drug-resistant seizures in Dravet syndrome and was reported to reduce convulsive-seizure frequency (Devinsky, et al., 2017, NEJM 376(21):2011-2020).

In cases with more drug resistant seizures, drugs such as topiramate, vigabatrin, and tiagabine, and/or a ketogenic diet are used as alternative treatments. Treatments also include cognitive rehabilitation through psychomotor and speech therapy. In addition, valproate is often administered to prevent recurrence of febrile seizures and benzodiazapine is used for long lasting seizures, but these treatments are usually insufficient.

Polypharmacy, the use of two or more anti-epileptic drugs, for the treatment of epilepsy or epileptic encephalopathy (e.g., Dravet syndrome or Lennox-Gastaut syndrome) can result in a significant patient burden, as the side effects, or adverse events from the multiple medications can be additive, and result in limiting the effectiveness of the therapy due to intolerability; in other words the small benefit of a medication may not outweigh the risk or negative effects the drug is having on the patient.

### Dosing of Fenfluramine

Any effective dose of fenfluramine can be employed in the context of the present disclosure. In some embodiments, a daily dose of fenfluramine is less than about 10 mg/kg/day, such as less than about 10 mg/kg/day, less than about 9 mg/kg/day, less than about 8 mg/kg/day, less than about 7 mg/kg/day, less than about 6 mg/kg/day, less than about 5 mg/kg/day, less than about 4 mg/kg/day, less than about 3.0 mg/kg/day, less than about 2.5 mg/kg/day, less than about 2.0 mg/kg/day, less than about 1.5 mg/kg/day, or less than about 1.0 mg/kg/day, such as about 1.0 mg/kg/day, about 0.95 mg/kg/day, about 0.9 meg/kg/day, about 0.85 mg/kg/day, about 0.85 mg/kg/day, about 0.8 mg/kg/day, about 0.75 mg/kg/day, about 0.7 mg/kg/day, about 0.65 mg/kg/day, about 0.6 mg/kg/day, about 0.55 mg/kg/day, about 0.5 mg/kg/day, about 0.45 mg/kg/day, about 0.4 mg/kg/day, about 0.350 mg/kg/day, about 0.3 mg/kg/day, about 0.25 mg/kg/day, about 0.2 mg/kg/day, about 0.15 mg/kg/day to about 0.1 mg/kg/day, about 0.075 mg/kg/day, about 0.05 mg/kg/day, about 0.025 mg/kg/day, about 0.0225 mg/kg/day, about 0.02 mg/kg/day, about 0.0175 mg/kg/day, about 0.015 mg/kg/day, about 0.0125 mg/kg/day, or about 0.01 mg/kg/day is employed.

Put differently, a preferred dose is less than about 10 to about 0.01 mg/kg/day. In some cases the dose is less than about 10.0 mg/kg/day to about 0.01 mg/kg/day, such as less than about 5.0 mg/kg/day to about 0.01 mg/kg/day, less than about 4.5 mg/kg/day to about 0.01 mg/kg/day, less than about 4.0 mg/kg/day to about 0.01 mg/kg/day, less than about 3.5 mg/kg/day to about 0.01 mg/kg/day, less than about 3.0 mg/kg/day to about 0.01 mg/kg/day, less than about 2.5 mg/kg/day to about 0.01 mg/kg/day, less than about 2.0 mg/kg/day to about 0.01 mg/kg/day, less than about 1.5 mg/kg/day to about 0.01 mg/kg/day, or less than about 1.0 mg/kg/day to 0.01 mg/kg/day, such as less than about 0.9 mg/kg/day, less than about 0.8 mg/kg/day, less than about less than about 0.7 mg/kg/day, less than about 0.6 mg/kg/day to about 0.01 mg/kg/day, less than about 0.5 mg/kg/day to about 0.01 mg/kg/day, less than about 0.4 mg/kg/day to about 0.01 mg/kg/day, less than about 0.3 mg/kg/day to about 0.01 mg/kg/day, or less than about 0.2 mg/kg/day to about 0.01 mg/kg/day.

The present disclosure encompasses a recognition that surprisingly low doses of fenfluramine are therapeutically effective, particularly for inhibiting or eliminating seizures in epilepsy patients. In some embodiments, a daily dose of less than about 2.5 mg/kg/day, less than about 2.0 mg/kg/day, less than about 1.5 mg/kg/day, or less than about 1.0 mg/kg/day, such as about 1.0 mg/kg/day, about 0.95 mg/kg/day, about 0.9 meg/kg/day, about 0.85 mg/kg/day, about 0.85 mg/kg/day, about 0.8 mg/kg/day, about 0.75 mg/kg/day, about 0.7 mg/kg/day, about 0.65 mg/kg/day, about 0.6 mg/kg/day, about 0.55 mg/kg/day, about 0.5 mg/kg/day, about 0.45 mg/kg/day, about 0.4 mg/kg/day, about 0.350 mg/kg/day, about 0.3 mg/kg/day, about 0.25 mg/kg/day, about 0.2 mg/kg/day, about 0.15 mg/kg/day to about 0.1 mg/kg/day, about 0.075 mg/kg/day, about 0.05 mg/kg/day, about 0.025 mg/kg/day, about 0.0225 mg/kg/day, about 0.02 mg/kg/day, about 0.0175 mg/kg/day, about 0.015 mg/kg/day, about 0.0125 mg/kg/day, or about 0.01 mg/kg/day is employed.

In some preferred embodiments, a dose of fenfluramine is within a range of about 2.5 to about 0.1 mg/kg/day. In some embodiments, a dose of fenfluramine is less than about 2.5 mg/kg/day to about 0.1 mg/kg/day, such as less than about 2.5 mg/kg/day to about 0.1 mg/kg/day, less than about 2.0 mg/kg/day to about 0.1 mg/kg/day, less than about 1.5 mg/kg/day to about 0.1 mg/kg/day, or less than about 1.0 mg/kg/day to 0.1 mg/kg/day, such as less than about 0.9 mg/kg/day, less than about 0.8 mg/kg/day, less than about less than about 0.7 mg/kg/day, less than about 0.6 mg/kg/day to about 0.01 mg/kg/day, less than about 0.5 mg/kg/day to about 0.01 mg/kg/day, less than about 0.4 mg/kg/day to about 0.1 mg/kg/day, less than about 0.3 mg/kg/day to about 0.1 mg/kg/day, or less than about 0.2 mg/kg/day to about 0.1 mg/kg/day.

As indicated above the dosing is based on the weight of the patient. However, for convenience the dosing amounts may be preset such as in the amount of 1.0 mg, 2.5 mg, 5 mg, 10 mg, 15 mg, 20 mg, 30 mg, 40 mg, or 50 mg. In certain instances, the dosing amount may be preset such as in the amount of about 0.25 mg to about 5 mg, such as about 0.25 mg, about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5.0 mg.

In general the smallest dose which is effective should be used for the particular patient.

The present disclosure encompasses a recognition that in embodiments where fenfluramine is administered at a low dose (e.g., within a range of 0.1 mg/kg to 0.5 mg/kg, e.g., a dose of 0.1 mg/kg to 0.35 mg/kg twice daily), the antiseizure activity of fenfluramine may be susceptible to interference by other agents such as certain serotonin receptor antagonists. In certain embodiments, a dose of fenfluramine is increased when one or more certain serotonin-receptor antagonists are being co-administered.

The dosing amounts described herein may be administered one or more times daily to provide for a daily dosing amount, such as once daily, twice daily, three times daily, or four or more times daily, etc.

In certain embodiments, the dosing amount is a daily dose of 30 mg or less, such as 30 mg, about 29 mg, about 28 mg, about 27 mg, about 26 mg, about 25 mg, about 24 mg, about 23 mg, about 22 mg, about 21 mg, about 20 mg, about 19 mg, about 18 mg, about 17 mg, about 16 mg, about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg. In general the smallest dose which is effective should be used for the particular patient. In some cases, the dose is generally well below the dosing used in weight loss.

The dose of fenfluramine to be used in a method of the present invention can be provided in the form of a kit, including instructions for using the dose in one or more of the methods of the present invention. In certain embodiments, the kit can additionally comprise a dosage form comprising one or more co-therapeutic agents.

### Dosage Forms and Administration of Fenfluramine

The dose of fenfluramine administered according to the methods of the present invention can be administered systemically or locally. Methods of administration may include administration via enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, it may be desirable to administer one or more compounds of the invention locally to the area in need of treatment. This may be achieved, for example, by local infusion during, topical application, by injection, by means of a catheter, by means of a suppository, or by means of an implant, said implant being of a porous, non-porous, or gelatinous material, including membranes, such as sialastic membranes, or fibers.

In some embodiments, a dose of fenfluramine administered in the methods of the present invention can be formulated in any pharmaceutically acceptable dosage form including, but not limited to (a) oral dosage forms such as tablets including orally disintegrating tablets, capsules, and lozenges, oral solutions or syrups, oral emulsions, oral gels, oral films, buccal liquids, powder e.g. for suspension, and the like; (b) injectable dosage forms; (c) transdermal dosage forms such as transdermal patches, ointments, creams; (c) inhaled dosage forms; and/or (e) nasally, (f) rectally, (g) vaginally administered dosage forms.

Such dosage forms can be formulated for once a day administration, or for multiple daily administrations (e.g. 2, 3 or 4 times a day administration). Alternatively, for convenience, dosage forms can be formulated for less frequent administration (e.g., monthly, bi-weekly, weekly, every fourth day, every third day, or every second day), and formulations which facilitate extended release are known in the art.

In some embodiments, a dosage form of fenfluramine employed in the methods of the present invention can be prepared by combining fenfluramine or a pharmaceutically acceptable salt thereof with one or more pharmaceutically acceptable diluents, carriers, adjuvants, and the like in a manner known to those skilled in the art of pharmaceutical formulation.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient (fenfluramine), as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

For an oral solid pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers.

By way of illustration, the fenfluramine composition can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

Particular formulations of the invention are in an oral liquid form. The liquid can be a solution or suspension and may be an oral solution or syrup, which is included in a bottle with a syringe graduated in terms of milligram amounts which will be obtained in a given volume of solution. The liquid solution makes it possible to adjust the volume of solution for appropriate dosing of small children, who can be administered fenfluramine in an amount anywhere from 1.25 mg to 30 mg and any amount between in 0.25 milligram, increments and thus administered in amounts of 1.25 mg, 1.5 mg, 1.75 mg, 2.0 mg, etc.

In alternate embodiments, the dispensing device may be a syringe or graduated pipette useful for delivering varying doses of the fenfluramine liquid. In another embodiment, the dispensing device is a metered dosing device capable of dispensing a fixed volume of fenfluramine liquid. In some exemplary embodiments, a dose delivered by the metered dosing device is adjustable.

The formulation may be a solution or suspension and is prepared such that a given volume of the formulation contains a known amount of active fenfluramine.

For example, some embodiments, the dispensing device is a syringe is graduated in one milliliter increments and the liquid fenfluramine formulation is characterized such that one milliliter in volume of formulation includes precisely one milligram of fenfluramine. In this manner, the patient may be correctly dosed with a desired milligram dosage of fenfluramine based on a volume of liquid formulation administered to the patient orally.

In alternate embodiments, the dispenser is a syringe connected to the container and configured to withdraw the liquid formulation from the container, wherein the syringe is marked with levels of graduation noting volume of formulation withdrawn, or a metered dose dispenser for delivering a predetermined volume of the formulation to said patient, or a metered dispensing device calibrated to deliver a predetermined volume of the liquid, permitting convenient, consistent, and accurate dosing.

Fenfluramine can be administered in the form of the free base, or in the form of a pharmaceutically acceptable salt, for example selected from the group consisting of hydrochloride, hydrobromide, hydroiodide, maleate, sulphate, tartrate, acetate, citrate, tosylate, succinate, mesylate and besylate. Further illustrative pharmaceutically acceptable salts can be found in Berge et al., J. Pharm Sci. (1977) 68(1): 1-19.

Fenfluramine for use in the methods of the present invention may be produced according to any pharmaceutically acceptable process known to those skilled in the art. Examples of processes for synthesizing fenfluramine are provided in the following documents: U.S. Pat. Nos. 10,351,509, 10,351,510, GB1413070, GB1413078 and EP441160.

### Co-Therapies

In some embodiments of the kits or methods described herein, fenfluramine can be employed as a co-therapy in the treatment of epilepsy. Fenfluramine can be co-administered in combination with one or more pharmaceutically active agents, which may be provided together with the fenfluramine in a single dosage formulation, or separately, in one or more separate pharmaceutical dosage formulations. Where separate dosage formulations are used, the subject composition and ore or more additional agents can be administered concurrently, or at separately staggered times, i.e., sequentially.

In some embodiments, the agents are co-therapeutic agents, such as anticonvulsants. Suitable co-therapeutic agents can be selected from the group consisting of carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam. Use of a pharmaceutically acceptable salt of a co-therapeutic agent is also contemplated.

In some embodiments, fenfluramine is co-administered with an anti-psychotic selected from: phenothiazines (trifluoperazine, perphenazine, prochlorperazine, acetophenazine, triflupromazine, mesoridazine), butyrophenones (haloperidol), thioxanthenes (chlorprothixene), dihydroindoles (molindone), diphenylbutylpiperidines (pimozide), risperidone, quetiapine, aripiprazole, paliperidone, cariprazine, brexpiprazole, and tricyclic antihistamines (cyproheptadine; pizotifen; ketotifen, azatadine, loratadine and desloratadine).

The invention is further illustrated in the following Examples.

### VII. EXAMPLES

### Example 1: Fenfluramine Administration with Antiserotonergic Drugs in Maximal Electroshock Model of Epilepsy

The present example describes synthetic and/or contraindicative effects of various compounds in combination with fenfluramine. Specifically, the present example describes characterization of anticonvulsant effects of fenfluramine when administered in combination with various other compounds using a murine model of seizures: a Maximal Electroshock (IVIES) model. Maximal Electroshock (IVIES) is a model of acute generalized tonic clonic seizures. Induction of seizures is caused by 60 Hz 0.2 second alternating 50 mA current in the mouse. Tonic hindlimb extensions are measured to assess seizures or seizure protection.

FEN (alternatively, FFA) was administered 4 hours prior to testing in the MES test, with each of the investigational compounds then being administered to a cohort of mice at the specific time point so that testing coincided with the FEN time of peak activity (4 hours). So, for example, a compound with a 1 hour time of peak activity would be administered 3 hours after FEN administration in the study design.

Fenfluramine was dosed at MES ED50, so in simplistic terms, a compound that also exerts an anticonvulsant effect would increase the number of mice with seizures above 50% protected, whereas a compound that prevents fenfluramine's anticonvulsant effects will reduce the number of mice with seizures below 50% protected. A confounding factor in this model is drugs with antagonist activity at MAch receptors, and particularly muscarinic blockade at M1 which is known to interfere with the tonic hind limb extension in the MES model and so can mask effects of drugs at other receptors. [Bhattacharya S K, et al., Indian J Exp Biol. 1991 March; 29(3):237-40.]

One compound, doxepin, has been extensively characterized in mice in several acute and chronic seizure models. In the IVIES test, doxepin has a time of peak activity (TPE) of 5 min and an ED50 of 6.6 mg (IP), albeit they also reported an ED50 of 8.2 mg/kg at a 0.25 hr time post-administration.

TABLE 4

| Compound | | | Time | |
|---|---|---|---|---|
| Name and Test | | | to | Ki in nM |
| Number ID | Structure | Dose^{a} | peak^{b} | (unless noted)^{c} |
| perphenazine Trilafon ™ 1. | [chemical structure] | 1.4 | 1.0 | 5-HT_{1A} 421 5-HT_{ID} — 5-HT_{2A} 5.6 5-HT_{2C} 132 |
| mianserin Tolvon ™ 2. | [chemical structure] | 0.2 | 11.0 | 5-HT_{1A} 400-2600 5-HT_{1D} 220-400 5-HT_{2A} 1.6-55 5-HT_{2C} .63-6.5 |
| N-desmethyl- clozapine Active metabolite of Clozaril ™ 3. | [chemical structure] | 5.3 | 11.0 | 5-HT_{1A} 160 5-HT_{1D} 130 5-HT_{2A} 2.59 5-HT_{2C} 4.8 |
| doxepin Sinequan^{TM} Silenor ™ 4. | [chemical structure] | 16.4 | 00.25 | 5-HT_{1A} 276-868 5-HT_{1D} 5-HT_{2A} 11-27 5-HT_{2C} 24-46 M1 MAchR 38 |
| asenapine Saphris ™ 5. | [chemical structure] | 0.6 | 11.0 | 5-HT_{1A} 15 5-HT_{1D} 10.2 5-HT_{2A} 0.77 5-HT_{2C} 0.27 |
| clomipramine Anafranil ™ 6. | [chemical structure] | 50 | 11 | 5-HT_{1A} >7000 5-HT_{1D} >10,000 5-HT_{2A} 27-35.5 5-HT_{2C} 64.6 M1 MAchR 37 |
| Carbamazepine Tegretaol ™ 7. | [chemical structure] | 20 | 0.25 | Sodium channel blocker, weak indirect serotonin releaser |
| Chlorpromazine Thorazine ™ Largactil ™ 8. | [chemical structure] | 4 | 00.5 | 5-HT_{1A} 3115 5-HT_{1D} 452 5-HT_{2A} 3.715 5-HT_{2C} 15.85 |
| Pimozide Orap ™ 9. | [chemical structure] | 3 | 0.5 | 5-HT_{1A} 650 5-HT_{1D} 3100 5-HT_{2A} 3.175 5-HT_{2C} 3350 |
| Loxapine Adasuve ™ Loxapac ™ 10. | [chemical structure] | 20 | 0.5 | 5-HT_{1A} 2456 5-HT_{1B} 388 5-HT_{1D} 3468 5-HT_{2A} 3.175 5-HT_{2C} 9.5-21 |
| Pindolol Visken ™ 11. | [chemical structure] | 15 | 1 | 5-HT_{1A} 33.3 5-HT_{1D} 4900 5-HT_{2A} 9332 5-HT_{2C} 10,000 |
| Binospirone MDL-73,005-EF 12. | [chemical structure] | | | pIC50^{d} 5-HT_{1A} 8.6 5-HT_{1B} 4.5 5-HT1A antagonist at postsynaptic receptors and efficacious partial agonist at somato- dendritic autoreceptors |
| BRL 15572 13. | [chemical structure] | | | pKi 5-HT_{1A} 7.7 5-HT_{1D} 7.9 |
| Lamotrigine Lamictal ™ 14. | [chemical structure] | | | Sodium channel blocker |

^{a}Compound Dose (mg/kg, i.p.; 2x published ED50 or hightest dose reported evaluated in mice)
^{b}Time to Peak Activity
^{c}Values from ChemBL, PDSP or DrugBank.ca databases and published literature
^{d}pIC50 = log10 concentration giving 50% inhibition of specific binding [Moser, et al., Br. J. Pharmacol. (1990), 99, 343-349]

TABLE 5
Results of MES Test of Fenfluramine at 3 mg/kg tested with Serotonergic
Inhibitors. (All routes of administration: IP).

| | | | Type of Test: | |
|---|---|---|---|---|
| Compound ID: Fenfluramine | | | Combination Identification | |
| Test number | Dose | Time of | Impaired | Protected |
| ID | (mg/kg) | Test (hr) | (I/F) | (N/F) |
| 1 | 1.4 | 0.5 | 0/8 | 2/8 |
| 2 | 0.2 | 1 | 0/8 | 2/8 |
| 3 | 5.3 | 1 | 0/8 | 1/8 |
| 4 | 16.4 | 0.25 | 1/8 | 7/8 |
| 5 | 0.6 | 1 | 0/8 | 2/8 |
| 6 | 50 | 1 | 0/8 | 7/8 |
| VEH | N/A | 4 | 0/8 | 4/8 |
| 7. AED | 20 | 0.25 | 0/8 | 8/8 |
| 8 | 4 | 0.5 | 1/8 | 5/8 |
| 9 | 3 | 0.5 | 0/8 | 3/8 |
| 10 | 20 | 0.5 | 8/8 | 1/8 |
| 11 | 15 | 1.0 | 0/8 | 5/8 |
| 12 | 5 | 0.5 | 0/8 | 3/8 |
| 13 | 30 | 0.25 | 0/8 | 1/8 |
| 14 AED | 19 | 0.5 | 0/8 | 8/8 |
| VEH | 0.5% | N/A | 0/8 | 5/8 |
| | methylcelluose | | | |
| VEH alone | 0.9% saline | 4 | 0/8 | 0/8 |
| (no | | | | |
| fenfluramine) | | | | |

The results above demonstrate the loss of antiseizure effect when fenfluramine is administered with an inhibitor of various serotonin receptors. General inhibition varies by compound but includes inhibition of H1, muscarinic M1, 5-HT 2A and 2C, dopamine receptors, and monoamine transporters (serotonin, dopamine and norepinephrine). The loss of seizure protection was most pronounced with compounds that were potent antagonists at 5-HT_{2A} and 5-HT_{2C} receptors. Interpretation of the results with respect to Test compounds 4 and 6 is hampered nearly equivalent binding of these compounds at the M1 receptor as at the 5-HT_{2A} and 5-HT_{2C} receptors. Another interpretation is that these compounds were not sufficiently potent to occupy the 5-HT_{2A} and 5-HT_{2C} receptors at the doses administered, however the apparent additive effect to fenfluramine's seizure protection is most likely a result of the muscarinic blockade. Cyproheptadine and amitriptyline are also potent M1 receptor antagonists that make then unsuitable for this model.

### Example 2: Fenfluramine Administration with Antiserotonergic Drugs in MES Models of Epilepsy

The present example describes synthetic and/or contraindicative effects of various compounds in combination with fenfluramine in an MES model of epilepsy, using a procedure as described in Example 1 above. Various dosing was tested to establish a range of fenfluramine (FFA) that give different levels of seizure protest in this model. Results are depicted in Table 6 below.

TABLE 6

| | | Activity in MES model |
|---|---|---|
| Compound | Pathway | in presence of FFA |
| 3 mg/kg IP, 2 hr pre-MES. | SOW #1: MES Identification | FFA protected = 3/4 |
| FFA- original testing | | |
| 10 mg/kg IP FFA, 2 hr pre- | SOW #1: MES Time Course | FFA protected = 3/4 |
| MES. - original testing | | |
| 10 mg/kg IP FFA, 4 hr pre- | SOW #1: MES Time Course | FFA protected = 4/4 |
| MES. - original testing | | |
| 2.5 mg/kg IP FFA, 4 hr pre- | SOW #1: MES Quantification | FFA protected = 3/4 |
| MES. - original testing | | |
| 5 mg/kg IP FFA, 4 hr pre- | SOW #1: MES Quantification | FFA protected = 6/8 |
| MES. - original testing | | |
| 10 mg/kg IP FFA, 4 hr pre- | SOW #1: MES Quantification | FFA protected = 8/8 |
| MES. - original testing | | |
| SOW #2 (−) control: 3 mg/kg | (−) control | FFA protected = 4/8 |
| FFA, 4 hr pre-MES | | |
| SOW #3 (−) control: 3 mg/kg | (−) control | FFA protected = 5/8 |
| FFA, 4 hr pre-MES | | |

These data show that 10 mg/kg of FFA consistently provide 100% protection in the IVIES model. 3 mg/kg FFA protects 50-60% of animals in the MES assay. Accordingly, this dose was used for the subsequent experiments with FFA in combination with a variety of other agents as described in Table 7 below.

TABLE 7
Various compounds on anti-seizure activity of FFA in MES models

| | | Activity in MES model |
|---|---|---|
| Compound | Pathway | in presence of FFA |
| Doxepin | Serotonin-norepinephrine | FFA protected = 7/8 |
| | reuptake inhibitor (SNRI) | |
| | high affinity antagonist of the | |
| | histamine H1 receptor (Ki <1 nM) | |
| | M1 antagonist (Ki ~= 18 nM) | |
| | 5-HT2a IC50 = 38 nM | |
| | 5-HT2a IC50 = 46 nM | |
| Clomipramine | Serotonin-norepinephrine | FFA protected = 7/8 |
| | reuptake inhibitor (SNRI) | |
| | ED(50) values showed a ratio | |
| | of anticholinergic effect to | |
| | antidepressant activity was | |
| | <0.8 | |
| Chlorpromazine | Antagonist of 5-HT2A, B, C, 5- | FFA protected = 5/8 |
| | HT6 and 5-HT7 | |
| Pimozide | Antagonist of 5-HT2A and 5- | FFA protected = 3/8 |
| | HT7 | |
| Pindolol | Nonselective beta blocker | FFA protected = 5/8 |
| | Not reported as active on 5-HT | |
| | receptor | |
| Binospirone (MDL 73005EF) | Agonist of the serotonin 5- | FFA protected = 3/8 |
| | HT1A receptor | |
| Clozapine | Antagonist of 5-HT2A | FFA reduced protection = 1/8 |
| | Some evidence it binds to a | |
| | GABA-B receptor(s) | |
| | Black box warning for seizure risk | |
| BRL 15572 | Selective antagonist for 5- | FFA reduced protection = 1/8 |
| | HT1D | |
| N-Desmethylclozapine | Metabolite of Clozapine | FFA reduced protection = 1/8 |
| | Antagonist of 5-HT2A | |
| | Also reported as an allosteric | |
| | agonist at the Ml and δ-opioid | |
| | receptors, but less M1 activity | |
| | than clozapine itself | |
| Loxapine | Antagonist of 5-HT2A | FFA reduced protection = 1/8 |
| | Also reported as a 5-HT2C | |
| | antoginist | |
| Perphenazine | Antagonist of 5-HT2A, 5-HT6 | FFA reduced protection = 2/8 |
| | and 5-HT7 | |
| Mianserin | Antagonist of 5-HT2A and 5- | FFA reduced protection = 2/8 |
| | HT2C | |
| | Reported activity at other 5-HT | |
| | receptors | |
| Asenapine | Inverse agonist at 5-HT1A | FFA reduced protection = 2/8 |
| | Extremely potent Antagonist at | |
| | 5-HT2A, 5-HT2C and | |
| | antagonist at 5-HTIB, 5- | |
| | HT2B, 5-HT5A, 5-HT6, and 5- | |
| | HT7 receptors | |
| Lamotrigine | Sodium channel blocker; may | Protected = 8/8 |
| | act presynaptically on voltage- | |
| | gated sodium channels to | |
| | decrease glutamate release | |
| | May also antagonize voltage- | |
| | gated Ca2+ channels and the 5- | |
| | HT3 receptor | |
| Carbamazepine | Antagonist of voltage-gated | Protected = 8/8 |
| | sodium channels | |
| | Some evidence it is a 5-HT | |
| | releasing agent/re-uptake | |
| | blocker | |

As described in the above table, clozapine, BRL 15572, N-desmethylclozapine, and loxapine all appear to block FFA anti-seizure activity in a MES model. In addition, perphenazine, mianserin, and asenapine may also block FFA anti-seizure activity in a MES model. In contrast, Doxepin, Clomipramine, Chlorpromazine, Pimozide, and Pindolol do not block FFA anti-seizure activity. Additionally, no interference was observed of FFA activity on lamotrigine or carbamazinpine in IVIES model.

FIG. 2 provides a summary table of inhibitory activity of different 5-HT receptor antagonists on different 5-HT receptors, and the corresponding effect on FFA activity in the IVIES model.

### Example 3: Fenfluramine Administration with Antiserotonergic Drugs in Zebrafish Models of Epilepsy

The present example describes synthetic and/or contraindicative effects of various compounds in combination with fenfluramine in a zebrafish model of epilepsy.

Zebrafish embryos (Danio rerio) were housed under standard aquaculture conditions. Experiments were carried out using protocols consistent with those described in U.S. Patent Publication No. 20180325909.

Results are summarized in the table depicted in FIG. 3. The number of astericks indicates potency, with *** representing the highest potency and * the lowest potency. As provided in FIG. 3, 5-HT1A is not active in a Faingold-SUDEP model or a DeWitt-Dravel zebrafish model of locomotion, while 5-HT1D shows antagonist activity in both a DeWitt-Dravel zebrafish model of locomotion and of epilepitform EEG. Similarly, 5-HT2A and 2C blocked fenfluramin anti-confulsant activity in a Faingold-SUDEP model and both DeWitt-Dravel zebrafish models (hyperlocomotion and epileptiform EEG).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of treating epilepsy, the method comprising:
administering a therapeutically effective amount of fenfluramine or a pharmaceutically acceptable salt thereof to a patient in need thereof, wherein the patient is not concurrently being administered a serotonin (5-HT)-receptor antagonist.

2. The method of claim 1, wherein the patient is also characterized by extreme weight loss, wasting, cachexia and/or loss of appetite.

3. The method of claim 1, wherein the patient is also characterized by a co-morbid psychiatric condition or psychosis.

4. (canceled)

5. The method of claim 1, further comprising:
administering a therapeutically effective amount of fenfluramine or a pharmaceutically acceptable salt thereof,
monitoring the patient for extreme weight loss, wasting, cachexia and/or loss of appetite.

6. The method of claim 2, further comprising:
administering an appetite stimulant that is not a serotonin (5-HT)-receptor antagonist.

7. (canceled)

8. The method of claim 1, further comprising:
providing to the patient instructions that the anti-seizure efficacy of fenfluramine or a pharmaceutically acceptable salt thereof may be reduced by administration of a serotonin receptor antagonist.

9. (canceled)

10. The method of claim 1, wherein the serotonin receptor antagonist is selected from: cyproheptadine, or a 5-HT_{1A} serotonin receptor antagonist, a 5-HT_{1D} serotonin receptor antagonist, a 5-HT_{2A} serotonin receptor antagonist, or a 5-HT_{2C} serotonin receptor antagonist.

11. The method of claim 1, wherein the serotonin receptor antagonist is a 5-HT_{1A} serotonin receptor antagonist and/or 5-HT_{2C} serotonin receptor antagonist.

12. The method of claim 1, further comprising:
providing to the patient instructions that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

13. A method of treating a population of patients diagnosed with epilepsy and/or epileptic encephalopathy, the method comprising:
administering a therapeutically effective amount of fenfluramine or a pharmaceutically acceptable salt thereof,
monitoring the patients for extreme weight loss, wasting, cachexia and/or loss of appetite, and
in those patients that develop extreme weight loss, wasting, cachexia and/or loss of appetite, administering an appetite stimulant, wherein the appetite stimulant is a 5-HT_{1A} serotonin receptor antagonist or a 5-HT_{2C} serotonin receptor antagonist.

14. The method of claim 13, wherein for those patients to be administered an appetite stimulant, providing instructions that the anti-seizure efficacy of fenfluramine or a pharmaceutically acceptable salt thereof may be reduced by administration of a 5-HT_{1D} serotonin receptor antagonist or a 5-HT_{2A} serotonin receptor antagonist.

15. The method of claim 13, wherein the epilepsy and/or epileptic encephalopathy is or comprises Dravet syndrome, Lennox Gastaut syndrome, Rett syndrome, Doose syndrome, and/or West syndrome.

16. The method of claim 15, wherein the 5-HT_{1A} serotonin receptor antagonist and/or 5-HT_{2C} serotonin receptor antagonist is selected from cyproheptadine, clozapine, doxepin, quetiapine, ketotifen, pizotifen, perphenazine, mianserin, mirtazapine, risperidone and asenapine.

17. The method of claim 1, wherein the fenfluramine is administered in a dose that is in a range of from 1 mg/kg/day to 0.01 mg/kg/day up to a maximum dose of 26 mg/day.

18. The method of claim 1, wherein the fenfluramine administered in a dosage form selected from the group consisting of oral, injectable, transdermal, inhaled, nasal, buccal, rectal, vaginal and parenteral delivery.

19. The method of claim 18, wherein the dosage form is an oral solution and the fenfluramine is in an amount selected from the group consisting of 120 mg or less, 60 mg or less, 30 mg or less, and 20 mg or less.

20. The method of claim 1, further comprising:
administering a co-therapeutic agent selected from the group consisting of stiripentol, clobazam, and valproate.

21.-26. (canceled)

27. The method of claim 13, wherein the fenfluramine or a pharmaceutically acceptable salt thereof is administered in a dose that is in a range of from 10.0 mg/kg/day to 0.01 mg/kg/day.

28. The method of claim 13, wherein the fenfluramine or a pharmaceutically acceptable salt thereof is administered in an amount selected from the group consisting of 120 mg or less, 60 mg or less, 30 mg or less, and 20 mg or less.

29. A kit, comprising:
a container comprising a fenfluramine formulation, and
a package insert, a label or a medication guide comprising content warning against co-administration with a serotonin (5-HT)-receptor antagonist.

30. The kit of claim 29, wherein the 5-HT receptor antagonist is an antagonist at one or more of subtypes chosen from 5-HT_{1A}, 5-HT_{2A} and 5-HT_{2C}.

31. The kit of claim 29, wherein the 5-HT receptor antagonist is a 5-HT_{1D} serotonin receptor antagonist or a 5-HT_{2A} serotonin receptor antagonist.

32. The kit of claim 29, wherein the 5-HT receptor antagonist is 5-HT_{2A} and/or 5-HT_{2C} receptor antagonist that is selected from cyproheptadine, clozapine, doxepin, quetiapine, ketotifen, pizotifen, perphenazine, mianserin, mirtazapine, risperidone and asenapine.

33. The kit of claim 29, wherein the fenfluramine formulation is a liquid formulation.

34. The kit of claim 29, wherein the package insert, a label or a medication guide further informs that (a) fenfluramine can be used to treat Dravet syndrome or Lennox-Gastaut syndrome, and/or (b) fenfluramine treatment may result in weight loss, wasting, and/or loss of appetite.

35. (canceled)

36. The kit as claimed in claim 29, wherein:
the formulation is an oral solution comprising 2.5 milligram of fenfluramine in each milliliter of liquid solution; and
the instructions indicate dosing the patient based on patient weight and volume of oral solution administered.

37. The kit as claimed in claim 29, wherein the formulation is a solid oral formulation selected from the group consisting of: a tablet, a disintegrating table, a capsule, a lozenge, and a sachet.

38. The kit as claimed in claim 29, wherein said formulation is provided in a transdermal patch.
